(19) United States
(12) Patent Application Publication
(10) Pub. No.: US 2023/0374124 A1
(43) Pub. Date: Nov. 23, 2023

(54) COMPOSITIONS FOR TREATMENT ALOPECIA AREATA, BIOMARKERS FOR TREATMENT SUCCESS AND, METHODS OF USE THEREOF

(71) Applicant: ICAHN SCHOOL OF MEDICINE AT MOUNT SINAI, New York, NY (US)

(72) Inventor: Emma Guttman, New York, NY (US)

(21) Appl. No.: 18/030,983

(22) PCT Filed: Oct. 8, 2021
(86) PCT No.: PCT/US21/54095

§ 371 Date: Apr. 7, 2023

Related U.S. Application Data
(60) Provisional application No. 63/154,861, filed on Mar. 1, 2021.
(60) Provisional application No. 63/124,110, filed on Dec. 11, 2020.
(60) Provisional application No. 63/089,440, filed on Oct. 8, 2020.

(51) Int. Cl. C07K 16/24; A61K 45/06; A61P 17/14; G01N 33/68

(52) U.S. Cl. CPC C07K 16/247; A61K 45/06; A61P 17/14; G01N 33/6854; G01N 2500/20; G01N 2800/52

(57) ABSTRACT

The present disclosure is based, at least in part, on the discovery that the likelihood of improving severity of hair loss in a subject in need of at least one treatment for alopecia areata can be determined, in part, by measuring concentration of total IgE in a serum sample collected from the subject. Provided herein are compositions and methods for improving severity of hair loss in subjects having alopecia areata with elevated IgE and general treatment of alopecia areata by use of compositions having at least one treatment for alopecia areata.

### CROSS REFERENCE TO RELATED APPLICATIONS

The present application is a National Stage application of International Application No. PCT/US21/54095, filed Oct. 8, 2021, and published as WO WO2022076771 on Apr. 14, 2022, which claims priority to U.S. Provisional Application Nos. 63/089,440 filed Oct. 8, 2020; 63/124,110 filed Dec. 11, 2020; and 63/154,861 filed Mar. 1, 2021. These applications are incorporated herein by reference in their entirety for all purposes.

### FIELD

The present disclosure is based, at least in part, on the discovery that administering an antibody or antigen-binding fragment thereof that specifically binds an interleukin-4 receptor (IL-4R) can improve the severity of hair loss in a subject that has or is suspected of having alopecia areata (AA). The antibody or antigen-binding fragment thereof that specifically binds IL-4R includes, for example, dupilumab. Accordingly, provided herein are methods for treating AA in a subject by administering to a subject in need thereof an effective amount of antibody or antigen-binding fragment thereof that specifically binds IL-4R compound.

The present disclosure is also based, at least in part, on the discovery that the likelihood of improving severity of hair loss in a subject in need of at least one treatment for AA can be determined by measuring the concentration of total IgE in a serum sample collected from the subject. Accordingly, provided herein are methods for improving severity of hair loss in subjects having AA with elevated serum IgE and methods of general treatment of AA by use of compositions having at least one treatment for AA, such as steroids, immunosuppressive agents, potassium channel activators, janus kinase (JAK) inhibitors (JAK1, JAK2, JAK3 or any combination of JAKs), Tyrosine Kinase (TYK) inhibitors, interleukin-4 (IL-4) blockers, interleukin-4 receptor (IL-4R) blockers, interleukin 5 (IL-5) blockers, interleukin 5 receptor (IL-5R) blockers, interleukin 12/23 p 40 (IL-12/23) blockers, interleukin 13 (IL-13) blockers, interleukin 13 receptor (IL-13R) blockers, interleukin 23 (IL-23) blockers, type II helper T cell (Th2) cytokine inhibitors, modulators of sphingosine-1-phosphate (S1P), modulators of natural killer group 2 member D (NKG2D), modulators of thymic stromal lymphopoietin (TSLP), or any combination thereof.

### BACKGROUND

Alopecia areata (AA) is a chronic, relapsing autoimmune disorder with an approximate 2% lifetime incidence among patients in the United States, affecting both adult and pediatric populations, with no sex predilection. It often presents with an unpredictable course, ranging from partial (patchy AA) to complete scalp hair loss (alopecia totalis [AT]) or to total scalp, face, and body hair loss (alopecia universalis [AU]). Although patients with limited AA frequently undergo spontaneous resolution, those with a moderate-to-severe phenotype often have more recalcitrant disease, which can significantly impact their quality of life.

To date, there are no safe and effective treatments for patients with moderate-to-severe AA. Available therapies, including intralesional steroid injections, contact sensitization, and systemic immunosuppressants, show limited efficacy, are inconvenient for patients, and/or are unsuitable for long-term use. Thus, there is an unmet need for improved treatments for patients with AA.

### SUMMARY OF THE INVENTION

The present disclosure is based on, at least in part, the surprising discovery that Th2 cytokines and chemokines, such as IL-13, IL-4, CCL13, and CCL18, are upregulated in subjects with alopecia areata (AA). Up regulation of the Th2 axis was known to occur in subjects with other atopic diseases, such as atopic dermatitis (AD), but it was not accepted in the field that the Th2 axis was also upregulated in AA. The present disclosure encompasses compositions, methods and dosing regimens for treating AA using an antibody that targets the interleukin-4 receptor (IL-4R) based on the unexpected findings provided herein that administering dupilumab once a week promotes hair regrowth in AA subjects. The present disclosure also encompasses methods and dosing regimens using the concentration of total IgE in a serum sample collected from the subject as a biomarker to predict the likelihood of success in treating AA.

In some embodiments, methods herein can include administering to a subject in need thereof an effective amount of an antibody or an antigen-binding fragment thereof, wherein the antibody or antigen-binding fragment thereof specifically binds an interleukin-4 receptor (IL-4R), wherein the subject may have or may be suspected of having alopecia areata. In some embodiments, the antibody or antigen-binding fragment thereof that specifically binds an interleukin-4 receptor (IL-4R) can be formulated in a pharmaceutical composition, which can further comprise one or more pharmaceutically acceptable carriers. In some embodiments, an antibody or antigen-binding fragment thereof that specifically binds an interleukin-4 receptor (IL-4R) as disclosed herein can be dupilumab.

In some embodiments, methods herein of administering an antibody or antigen-binding fragment thereof that specifically binds an interleukin-4 receptor (IL-4R) can include administration of the antibody or antigen-binding fragment thereof to a subject that is a human patient having alopecia areata. In some embodiments, a subject herein can be a human adult patient having alopecia areata. In some embodiments, a subject herein can be a human child patient having alopecia areata.

In some embodiments, methods herein can include administering an antibody or antigen-binding fragment thereof that specifically binds an interleukin-4 receptor (IL-4R) to a subject at a single initial dose having about 100 mg/kg to about 600 mg/kg of the antibody or antigen-binding fragment thereof. In some embodiments, the subject is a human adult and the initial dose is about 600 mg/kg. In some embodiments, the subject is a human child and the initial dose is about 100 mg/kg to about 300 mg/kg.

In some embodiments, methods herein can include administration of a single initial dose that can be followed by one or more secondary doses comprising the antibody or antigen-binding fragment thereof that specifically binds an interleukin-4 receptor (IL-4R) disclosed herein. In some embodiments, secondary doses as disclosed herein can have about 100 mg/kg to about 600 mg/kg of the antibody or antigen-binding fragment thereof that specifically binds an interleukin-4 receptor (IL-4R). In some embodiments, a first secondary dose comprising the antibody or antigen-binding fragment thereof specifically that binds an interleukin-4 receptor (IL-4R) disclosed herein can be administered between about 5 days to about 10 days immediately preceding the initial dose. In some embodiments, each subsequent secondary dose comprising the antibody or antigen-binding fragment thereof specifically that binds an interleukin-4 receptor (IL-4R) disclosed herein can be administered by a schedule ranging from once every 5 days to once every 10 days.

In some embodiments, methods herein can include administering an antibody or antigen-binding fragment thereof specifically binds an interleukin-4 receptor (IL-4R) disclosed herein to a subject topically, systemically, subcutaneously, intravenously, or intranasally.

In some embodiments, methods herein can include administering an antibody or antigen-binding fragment thereof specifically binds an interleukin-4 receptor (IL-4R) disclosed herein to subject having or suspected of having alopecia areata and/or having partial to complete hair loss to scalp, face, body or any combination thereof.

In some embodiments, methods disclosed herein can include administering an antibody or antigen-binding fragment thereof specifically binds an interleukin-4 receptor (IL-4R) disclosed herein to a subject who has undergone or may be undergoing another therapy for alopecia areata. In some embodiments, another therapy for alopecia areata can include administration of corticosteroids, immunosuppressive agents (e.g., cyclosporine A), potassium channel activators, janus kinase (JAK) inhibitors, Tyrosine Kinase (TYK) inhibitors, interleukin 5 (IL-5) blockers, interleukin 5 receptor (IL-5R) blockers, interleukin 12/23 p 40 (IL-12/23) blockers, interleukin 13 (IL-13) blockers, interleukin 13 receptor (IL-13R) blockers, interleukin 23 (IL-23) blockers, Th2 cytokine inhibitors (e.g., including but not limited to OX40 (also known as CD134 or TNFRSF4), OX40 ligand (OX40L), modulators of sphingosine-1-phosphate (S1P), modulators of NKG2D, and modulators of thymic stromal lymphopoietin (TSLP)), or any combination thereof.

In some embodiments, methods herein can include measuring the levels of IgE in a subject having or suspected of having alopecia areata. In some examples, serum levels of IgE can be assessed prior to administration of the initial dose of the antibody or antigen-binding fragment thereof that specifically binds an interleukin-4 receptor (IL-4R). In some examples, serum levels of IgE can be assessed prior to administration of a secondary dose of the antibody or antigen-binding fragment thereof that specifically binds an interleukin-4 receptor (IL-4R). In some examples, serum levels of IgE can be assessed at any time during administration of the antibody or antigen-binding fragment thereof that specifically binds an interleukin-4 receptor (IL-4R) disclosed herein.

In some embodiments, methods herein can include administering to a subject in need thereof an effective amount of an antibody or an antigen-binding fragment thereof disclosed herein, wherein the antibody or antigen-binding fragment thereof specifically binds an interleukin-4 receptor (IL-4R), wherein the subject may have or may be suspected of having alopecia areata and/or the subject has a total IgE serum equal to or greater than about 200 IU/ml.

In some embodiments, methods herein can include measuring the levels of IgE in a subject having or suspected of having alopecia areata. In some embodiments, serum levels of IgE can be assessed prior to administration of the initial dose of an antibody or antigen-binding fragment thereof that specifically binds an interleukin-4 receptor (IL-4R) as disclosed herein. In some embodiments, serum levels of IgE can be assessed prior to administration of a secondary dose of an antibody or antigen-binding fragment thereof that specifically binds an interleukin-4 receptor (IL-4R) as disclosed herein. In some embodiments, serum levels of IgE can be assessed at any time during administration of the antibody or antigen-binding fragment thereof that specifically binds an interleukin-4 receptor (IL-4R) as disclosed herein. In some embodiments, serum levels of IgE can be measured using at least one electrochemiluminescence immunoassay.

Aspects of the present disclosure provide for methods of determining the likelihood of improving severity of hair loss in a subject in need thereof. In some embodiments, methods for determining the likelihood of improving severity of hair loss in a subject in need of at least one treatment for alopecia areata can include any of the following steps: (a) determining a concentration of a total IgE in a serum sample collected from the subject in need of at least one treatment for alopecia areata; (b) comparing the concentration of total IgE in the serum collected from the subject in need of at least one treatment for alopecia areata to a concentration of total IgE in serum samples collected from a population of normal healthy control subjects; and (c) determining a high likelihood of improving severity of hair loss in the subject in need of at least one treatment for alopecia areata when the total IgE in the serum collected from the subject in need thereof is elevated above a normal healthy concentration of total IgE, wherein the normal healthy concentration of total IgE is the concentration of total IgE in serum samples collected from the population of normal healthy control subjects. In some embodiments, a normal healthy concentration of total IgE can be less than 200 IU/ml.

In some embodiments, methods herein can determine the likelihood of improving severity of hair loss in a subject in need of at least one treatment for alopecia areata wherein the treatment for alopecia areata can be administration of a steroid, an immunosuppressive agent, a potassium channel activator, a janus kinase (JAK) inhibitor, a Tyrosine Kinase (TYK) inhibitor, an interleukin-4 (IL-4) blocker, an interleukin-4 receptor (IL-4R) blocker, an interleukin 5 (IL-5) blocker, an interleukin 5 receptor (IL-5R) blocker, an interleukin 12/23 p 40 (IL-12/23) blocker, an interleukin 13 (IL-13) blocker, an interleukin 13 receptor (IL-13R) blocker, an interleukin 23 (IL-23) blocker, a type II helper T cell (Th2) cytokine inhibitor, a modulator of Sphingosine-1-phosphate (S1P), a modulator of natural killer group 2 member D (NKG2D), a modulator of thymic stromal lymphopoietin (TSLP), or any combination thereof. In some embodiments, methods herein can determine the likelihood of improving severity of hair loss in a subject in need of at least one treatment for alopecia areata wherein the treatment can be administration of cortisone, cyclosporine A, OX40, OX40 ligand (OX40L), abrocitinib, baricitinib, ritlecitinib, CTP 543, ustekinumab, tralokinumab, lebrikizumab, nemolizumab, upadacitinib, tofacitinib, ruxolitinib, oclacitinib, peficitinib, fedratinib, filgotinib, cerdulatinib, gandotinib, lestaurtinib, momelotinib, pacritinib, BMS-986165, dupilumab, pitrakinra, CBP 201, AK 120, pascolizumab, or any combination thereof.

In some embodiments, methods herein can determine the likelihood of improving severity of hair loss in a subject in need of at least one treatment for alopecia areata where the likelihood of improving severity of hair loss can be at least a 30% improvement from a baseline Severity of Alopecia Tool (SALT) score, wherein the baseline SALT score was measured before the subject was treated with at least one treatment for alopecia areata. In some embodiments, methods herein can determine the likelihood of improving severity of hair loss in a subject in need of at least one treatment for alopecia areata where the likelihood of improving severity of hair loss can be up to a 75% improvement from a baseline SALT score, wherein the baseline SALT score was measured before the subject was treated with at least one treatment for alopecia areata. In some embodiments, methods herein can determine the likelihood of improving severity of hair loss in a subject in need of at least one treatment for alopecia areata where the likelihood of improving severity of hair loss can be at least a 30% improvement from a baseline SALT score after about 8 to about 72 weeks of dupilumab treatment, wherein the baseline SALT score was measured before the subject was treated with at least one treatment for alopecia areata. In some embodiments, methods herein can determine the likelihood of improving severity of hair loss in a subject in need of at least one treatment for alopecia areata where the likelihood of improving severity of hair loss can be at least a 50% improvement from a baseline SALT score after about 12 to about 72 weeks of dupilumab treatment, wherein the baseline SALT score was measured before the subject was treated with at least one treatment for alopecia areata. In some embodiments, methods herein can determine the likelihood of improving severity of hair loss in a subject in need of at least one treatment for alopecia areata where the likelihood of improving severity of hair loss can be at least a 75% improvement from a baseline SALT score after about 20 to about 72 weeks of dupilumab treatment, wherein the baseline SALT score was measured before the subject was treated with at least one treatment for alopecia areata.

In some embodiments, methods herein of determining the likelihood of improving severity of hair loss in a subject to be treated with dupilumab can include one or more of the following: (a) determining a concentration of a total IgE in a serum sample collected from the subject to be treated with dupilumab; (b) comparing the concentration of total IgE in the serum collected from the subject to be treated with dupilumab to a concentration of total IgE in serum samples collected from a population of normal healthy control subjects; and (c) determining a high likelihood of improving severity of hair loss when the total IgE in the serum collected from the subject to be treated with dupilumab is elevated above a normal healthy concentration of total IgE, wherein the normal healthy concentration of total IgE is the concentration of total IgE in serum samples collected from the population of normal healthy control subjects. In some embodiments, a normal healthy concentration of total IgE can be less than 200 IU/ml. In some embodiments, the likelihood of improving severity of hair loss can be at least a 30% improvement from a baseline Severity of Alopecia Tool (SALT) score, wherein the baseline SALT score was measured before the subject was treated with dupilumab. In some embodiments, the likelihood of improving severity of hair loss can be up to a 75% improvement from a baseline SALT score, wherein the baseline SALT score was measured before the subject was treated with dupilumab. In some embodiments, the likelihood of improving severity of hair loss can be at least a 30% improvement from a baseline SALT score after 8 to 72 weeks of dupilumab treatment, wherein the baseline SALT score was measured before the subject was treated with dupilumab. In some embodiments, the likelihood of improving severity of hair loss can be at least a 50% improvement from a baseline SALT score after 12 to 72 weeks of dupilumab treatment, wherein the baseline SALT score was measured before the subject was treated with dupilumab. In some embodiments, the likelihood of improving severity of hair loss can be at least a 75% improvement from a baseline SALT score after 20 to 72 weeks of dupilumab treatment, wherein the baseline SALT score was measured before the subject was treated with dupilumab.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

FIG. 1 shows a schematic depicting the protocol used to perform the exemplary phase 2a, randomized, double-blind, multicenter study disclosed herein.

FIG. 2 shows a schematic depicting the Consolidated Standards of Reporting Trials (CONSORT) Chart wherein a total of 65 AA patients were enrolled, of whom 60 were enrolled and randomized 2:1 to dupilumab and placebo arms. Information was available from 51 patients for primary endpoint analysis (week 24), from 44 patients for secondary endpoint analysis after the open-label phase (week 48), and from 28 patients for the analysis of the follow-up period, up to week 60.

FIGS. 3A-3D shows graphs depicting a least-squares mean change in the severity of Alopecia Tool (SALT) Score (Baseline—Post Treatment) (FIG. 3A) and responder rates over the duration of the Study (Until Week 72) for SALT₃₀ responders (FIG. 3B), SALT₅₀ responders (FIG. 3C), and SALT₇₅ responders (FIG. 3D) where red, blue and purple represent drug, placebo arms, and dupilumab after placebo (open-label), respectively and */**/*** denote significant (P<0.05/0.01/0.001 respectively) difference in the dupilumab arm as compared to placebo treatment, dupilumab open-label versus placebo treatment in the placebo arm, and change from baseline at each time point in the dupilumab arm, and in the open-label phase of the placebo arm.

FIG. 4 shows graphs depicting stratification of patients by personal and family history of atopy. Least-squares mean change in the severity of alopecia tool (SALT) score over the duration of the study in patients with (left panel) and without (right panel) personal and/or family history of atopy. Red, blue and purple numbers represent number of patients in the dupilumab, placebo, and dupilumab following placebo arms, respectively, in corresponding weeks. Solid lines represent patients treated with dupilumab, dotted lines represent patients that were assessed on weeks 60 and 72 but were no longer treated with dupilumab after week 48. */**/*** denote significant (P<0.05/0.01/0.001 respectively) difference in the dupilumab arm as compared to placebo treatment (black), change from baseline at each time point in the dupilumab arm (red), in the open-label phase of the placebo arm (purple), and in the placebo treatment phase in the placebo arm (blue).

FIG. 5 shows images depicting scalp pictures of four patients in the drug arm of the exemplary study disclosed herein. Panels A-D and H-K show scalp hair at baseline, week 24, week 48, and week 72 visits in patients continuing treatment after week 48. Panels E-G and L-N show scalp hair at baseline, week 24, and week 48 visits.

FIGS. 6A-6F shows graphs depicting Pearson correlation of changes in SALT scores with baseline IgE levels (FIGS. 6A and 6B), baseline total serum IgE as a predictor of pesponse to dupilumab (FIG. 6C), and responder rates based on IgE levels at baseline (FIGS. 6D-6F). Correlation of patients in the dupilumab arm after completing 48 weeks of treatment (FIG. 6A), correlation of all patients treated with 24 weeks of dupilumab together (i.e., those completing week 24 in the drug arm with those completing week 24 to week 48 in the placebo arm, after switching to dupilumab) (FIG. 6B). False discovery rate (FDR)<0.05 for both A and B. Receiver operating characteristic area under the curve (AUC) analysis utilizing IgE as a predictor of SALT₅₀ response at week 24 (FIG. 6C), with IgE levels able to predict response in 83% of AA dupilumab-treated patients. Responder (SALT_{30/50/75}) rates in the entire drug arm, along with lines depicting only patients with high or low IgE (threshold: 2001 U/ml) in the drug arm (FIGS. 6D-6F). SALT, Severity of Alopecia Tool. *,**,***denote significant (p<0.05/0.01/0.001 respectively) difference in responder rates in patients with high versus low IgE at baseline.

FIGS. 7A-7C shows graphs depicting responder rates based on duration since last hair regrowth. SALT₃₀ responder (FIG. 7A), SALT₅₀ responder (FIG. 7B), and SALT₇₅ responder (FIG. 7C) rates in the entire drug arm (blue), along with lines depicting only patients with less than 3 years since last hair regrowth (red), 3-6 years since last hair regrowth (green), and more than 6 years since last hair regrowth in the drug arm (dark red). SALT, Severity of Alopecia Tool.

FIG. 8 shows a heatmap depicting differentially expressed immune genes in samples collected from subjects treated with placebo or dupilumab for 0, 12, 24, or 48 weeks.

FIG. 9 shows a heatmap depicting differentially expressed immune genes within the Th1 axis in samples collected from subjects treated with placebo or dupilumab for 0, 12, 24, or 48 weeks.

FIGS. 10A-10B show graphs depicting fold-changes of Th1 (FIG. 10A) and Th1 specific (FIG. 10B) immune mediators in samples collected from subjects treated with placebo or dupilumab for 0, 12, 24, or 48 weeks as measured by quantitative real-time PCR.

FIG. 11 shows a heatmap depicting differentially expressed immune genes within the Th2 axis in samples collected from subjects treated with placebo or dupilumab for 0, 12, 24, or 48 weeks.

FIG. 12 shows a graph depicting fold-changes of Th2/Th1 immune mediators in samples collected from subjects treated with placebo or dupilumab for 0, 12, 24, or 48 weeks as measured by quantitative real-time PCR.

FIG. 13 shows a graph depicting fold-changes of Th2 specific immune mediators in samples collected from subjects treated with placebo or dupilumab for 0, 12, 24, or 48 weeks as measured by quantitative real-time PCR.

FIG. 14 shows a heatmap depicting differentially expressed immune genes within the Th17 axis in samples collected from subjects treated with placebo or dupilumab for 0, 12, 24, or 48 weeks.

FIG. 15 shows a graph depicting fold-changes of Th17 specific immune mediators in samples collected from subjects treated with placebo or dupilumab for 0, 12, 24, or 48 weeks as measured by quantitative real-time PCR.

FIG. 16 shows a heatmap depicting differentially expressed genes associated with hair keratins in samples collected from subjects treated with placebo or dupilumab for 0, 12, 24, or 48 weeks.

FIGS. 17A-17B show graphs depicting fold-changes of hair protein associated genes in samples collected from subjects treated with placebo or dupilumab for 0, 12, 24, or 48 weeks as measured by quantitative real-time PCR.

FIG. 18A shows a graph depicting percent improvement in differentially expressed hair protein associated genes in samples collected from subjects treated with placebo or dupilumab for 0, 12, 24, or 48 weeks.

FIG. 18B shows a graph depicting percent improvement in differentially expressed hair protein associated genes in samples collected from subjects treated with placebo or dupilumab for 0, 12, 24, or 48 weeks wherein the subjects had either IgE≥200 or a history of atopy.

FIG. 19A shows a heatmap depicting differentially expressed genes in lesional versus normal skin collected from subjects treated with placebo or dupilumab for 0, 12, 24, or 48 weeks.

FIG. 19B shows a graph depicting percent improvement in differentially expressed genes in lesional versus normal skin collected from subjects treated with placebo or dupilumab for 0, 12, 24, or 48 weeks.

FIG. 19C shows a graph depicting the number of differentially expressed genes in lesional versus normal skin collected from subjects treated with placebo or dupilumab for 0, 12, 24, or 48 weeks.

FIG. 20A shows a heatmap depicting differentially expressed genes in lesional versus normal skin collected from subjects treated with placebo or dupilumab for 0, 12, 24, or 48 weeks wherein the subjects had either IgE≥200 or a history of atopy.

FIG. 20B shows a graph depicting percent improvement in differentially expressed genes in lesional versus normal skin collected from subjects treated with placebo or dupilumab for 0, 12, 24, or 48 weeks wherein the subjects had either IgE≥200 or a history of atopy.

FIG. 20C shows a graph depicting the number of differentially expressed genes in lesional versus normal skin collected from subjects treated with placebo or dupilumab for 0, 12, 24, or 48 weeks wherein the subjects had either IgE≥200 or a history of atopy.

### DETAILED DESCRIPTION OF THE INVENTION

Efforts to understand the mechanisms of alopecia areata (AA), which are primarily based on mice models as well as few human studies, generated the understanding that alopecia areata (AA) was mediated by CD4⁺ and CD8⁺ cytotoxic T-lymphocytes, resulting in damage to hair follicles. These studies suggested that the primary pathogenic immune axis driving AA was the Th1 and related cytokines and chemokines within the Th1 immune axis such IFNγ/IL-12/IL-23p40, CXCL10, IL-2, IL-15. A role for JAK inhibitors in treatment of AA was originally suggested by experiments in murine models of AA and in humans in which hair regrowth was observed following application of either JAK1/2 or JAK1/2/3 inhibitors or TYK inhibitors, with associated effects on type I cellular immunity. However, the JAK family, comprised of JAK1/2/3 and tyrosine kinase 2 (TYK2), modulates most inflammatory pathways via signal transduction, and is not specific to one axis. As such, JAK inhibitors are targeting multiple cytokines and immune pathways and cannot prove a primary Th1 pathogenesis of alopecia areata as predominately believed by those in the field.

The present disclosure is based, at least in part, on the discovery that administering an antibody or antigen-binding fragment thereof that specifically binds an interleukin-4 receptor (IL-4R) can improve the severity of hair loss in a subject that has or is suspected of having alopecia areata (AA). The antibody or antigen-binding fragment thereof that specifically binds IL-4R includes, for example, dupilumab. Aspects of the present disclosure are also based in part on the surprising discovery that measuring serum IgE levels in a subject that has or is suspected of having AA can predict if the subject will be responsive to administration of an effective amount of antibody or antigen-binding fragment thereof that specifically binds IL-4R.

Accordingly, provided herein are methods for treating AA in a subject by administering to a subject in need thereof an effective amount of antibody or antigen-binding fragment thereof that specifically binds IL-4R. Such methods can rely on administration of the antibody or antigen-binding fragment thereof that specifically binds IL-4R formulated in a pharmaceutical composition. For example, the treatment obtained from such methods would treat AA in a subject having high total serum IgE levels by administering an antibody or antigen-binding fragment thereof that specifically binds IL-4R formulated in a pharmaceutical composition in an effective amount as part of a treatment regimen topically, systemically, subcutaneously, intravenously, or intranasally.

### I. Definitions

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat”, “treating”, or the like, mean to alleviate symptoms, eliminate the causation of symptoms either on a temporary or permanent basis, or to prevent or slow the appearance of symptoms of the named disorder or condition.

Although any methods and materials similar or equivalent to those described herein can be used in the practice of the present invention, the preferred methods and materials are now described. All publications mentioned herein are incorporated herein by reference in their entirety.

### II. Interleukin-4 Receptor (IL-4R) Antibody

The methods of the present disclosure include administering to a subject in need thereof an antibody or antigen-binding fragment thereof that specifically binds an interleukin-4 receptor (IL-4R), for example human IL-4R.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-IL-4R antibody, encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion (e.g., chimeric antigen receptor or CAR), humanized antibodies, chimeric antibodies, diabodies, single domain antibody (e.g., a V_{H} only antibody such as a nanobody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. In some embodiments, an antibody herein, e.g., anti-IL-4R antibody, can include an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

In some embodiments, an anti-IL-4R antibody disclosed herein may be a full-length antibody, which contains two heavy chains and two light chains, each including a variable domain and a constant domain. In some embodiments, an anti-IL-4R antibody disclosed herein can be an antigen-binding fragment of a full-length antibody. Examples of binding fragments encompassed within the term “antigen-binding fragment” of a full length antibody can include (i) a Fab fragment, a monovalent fragment consisting of the V_{L}, V_{H}, C_{L} and C_{H}1 domains; (ii) a F(ab′)2 fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

In some embodiments, antibodies disclosed herein (e.g., anti-IL-4R antibodies) disclosed herein can be of a suitable origin, for example, murine, rat, or human. Such antibodies are non-naturally occurring, i.e., would not be produced in an animal without human act (e.g., immunizing such an animal with a desired antigen or fragment thereof or isolated from antibody libraries). In some embodiments, antibodies disclosed herein (e.g., anti-IL-4R antibodies) can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the anti-IL-4R antibodies herein can be human antibodies, which may be isolated from a human antibody library or generated in transgenic mice. In some embodiments, fully human antibodies herein can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Amgen, Inc. (Fremont, Calif.) and HuMAb-Mouse™ and TC Mouse™ from Medarex, Inc. (Princeton, N.J.). In some embodiments, antibodies herein may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the antibody library display technology, such as phage, yeast display, mammalian cell display, or mRNA display technology as known in the art can be used to produce human antibodies and antibody fragments in vitro, from immunoglobulin variable (V) domain gene repertoires from unimmunized donors.

In some embodiments, the anti-IL-4R antibodies disclosed herein may be humanized antibodies or chimeric antibodies. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. In general, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. In some embodiments, the anti-IL-4R antibodies disclosed herein may have one or more Fv framework region (FR) residues of the human immunoglobulin that are replaced by corresponding non-human residues. In some embodiments, anti-IL-4R antibodies disclosed herein may be a humanized antibody comprising residues that are found neither in the recipient antibody nor in the imported CDR or framework sequences, but are included to further refine and optimize antibody performance. In some embodiments, anti-IL-4R antibodies disclosed herein may be a humanized antibody may comprise substantially all of at least one, and typically two, variable domains, in which all or substantially all of the CDR regions correspond to those of a non-human immunoglobulin and all or substantially all of the FR regions are those of a human immunoglobulin consensus sequence. In some embodiments, anti-IL-4R antibodies disclosed herein may be a humanized antibody that can include at least a portion of an immunoglobulin constant region or domain (Fc), typically that of a human immunoglobulin. Antibodies herein may have Fc regions modified as described in WO 99/58572. Other forms of humanized antibodies herein may have one or more CDRs (one, two, three, four, five, or six) which are altered with respect to the original antibody, which are also termed one or more CDRs “derived from” one or more CDRs from the original antibody. Humanized antibodies may also involve affinity maturation. Methods for constructing humanized antibodies are also well known in the art. See, e.g., Queen et al., Proc. Natl. Acad. Sci. USA, 86:10029-10033 (1989).

In some embodiments, an anti-IL-4R antibody disclosed herein can be a chimeric antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

In some embodiments, anti-IL-4R antibodies described herein can specifically bind to a corresponding target antigen (e.g., IL-4R) or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (e.g., IL-4R) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some embodiments, anti-IL-4R antibodies disclosed herein may be an antibody that “specifically binds” to a target antigen or an epitope thereof and may not bind to other antigens or other epitopes in the same antigen (i.e., only baseline binding activity can be detected in a conventional method).

In some embodiments, anti-IL-4R antibodies disclosed herein may be dupilumab. In some embodiments, methods of the present disclosure include administering one or more anti-IL-4R antibodies (e.g., dupilumab) to a subject in need thereof

### III. Pharmaceutical Compositions

In some embodiments, any antibody or antigen-binding fragment thereof that specifically binds an IL-4R (e.g., dupilumab) as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

In some embodiments, pharmaceutical compositions to be used in the present methods can comprise pharmaceutically acceptable carriers, excipients, or stabilizers in the form of lyophilized formulations or aqueous solutions. Acceptable carriers, excipients, or stabilizers are nontoxic to recipients at the dosages and concentrations used. In some embodiments, acceptable carriers, excipients, and/or stabilizers for use herein may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some embodiments, a pharmaceutical composition described herein can encompass liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes may be extruded through filters of defined pore size to yield liposomes with the desired diameter.

In some embodiments, any antibody or antigen-binding fragment thereof that specifically binds an IL-4R (e.g., dupilumab) disclosed herein or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In some embodiments, a pharmaceutical composition described herein can be formulated in sustained-release format. Suitable examples of sustained-release preparations include, but are not limited to, semipermeable matrices of solid hydrophobic polymers containing the antibody, which matrices are in the form of shaped articles, e.g. films, or microcapsules. Non-limiting examples of sustained-release matrices suitable for use herein can include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid, and the like.

In some embodiments, pharmaceutical compositions disclosed herein to be used for in vivo administration can be sterile. In some embodiments, pharmaceutical compositions disclosed herein can be sterilized by, for example, filtration through sterile filtration membranes. In some embodiments, pharmaceutical compositions disclosed herein can be placed into a container having a sterile access port, for example, an intravenous solution bag or vial having a stopper pierceable by a hypodermic injection needle.

In some embodiments, pharmaceutical compositions disclosed herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient (e.g., any antibody or antigen-binding fragment thereof specifically binds an IL-4R, such as dupilumab) can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from about 0.1 to about 500 mg of the active ingredient of the present disclosure (e.g., an antibody or antigen-binding fragment thereof specifically binds an IL-4R, such as dupilumab). In some embodiments, where pharmaceutical compositions herein are tablets or pills, the composition can be coated or otherwise compounded to provide a dosage form affording the advantage of prolonged action. In some embodiments, where pharmaceutical compositions herein are tablets or pills, the tablet or pill can comprise an inner dosage and an outer dosage component, the latter being in the form of an envelope over the former. In some embodiments, the two components can be separated by an enteric layer that may serve to resist disintegration in the stomach and/or permit the inner component to pass intact into the duodenum and/or to be delayed in release. A variety of materials can be used for such enteric layers or coatings, including, but not limited to a number of polymeric acids and mixtures of polymeric acids with such materials as shellac, cetyl alcohol and cellulose acetate.

In some embodiments, pharmaceutical compositions disclosed herein can include one or more surface-active agents. Suitable surface-active agents for use herein can include, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent can have between 0.05 and 5% surface-active agent and can be between 0.1 and 2.5%. It is to be appreciated that other ingredients may be added, for example mannitol or other pharmaceutically acceptable vehicles, if necessary.

In some embodiments, pharmaceutical compositions disclosed herein can be an emulsion. Suitable emulsions for use herein may be prepared using commercially available fat emulsions, such as Intralipid™, Liposyn™, Infonutrol™, Lipofundin™ and Lipiphysan™. In some embodiments, the active ingredient (e.g., an antibody or antigen-binding fragment thereof specifically binds an IL-4R, such as dupilumab) may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It is to be appreciated that other ingredients may be added, for example glycerol or glucose, to adjust the tonicity of the emulsion. In some embodiments, emulsions herein can contain up to 20% oil, for example, between 5 and 20%. In some embodiments, emulsions herein can be fat emulsions. In some embodiments, fat emulsions herein can have fat droplets between about 0.1 and about 1.0 μm, particularly about 0.1 and about 0.5 μm, and have a pH in the range of about 5.5 to about 8.0. In some embodiments, emulsion compositions can be those prepared by mixing an antibody with Intralipid™ or the components thereof (soybean oil, egg phospholipids, glycerol and water).

In some embodiments, pharmaceutical compositions disclosed herein can be prepared for inhalation or insufflation. Such compositions for inhalation or insufflation may include solutions and suspensions in pharmaceutically acceptable, aqueous or organic solvents, or mixtures thereof, and powders. In some embodiments, liquid or solid compositions may contain suitable pharmaceutically acceptable excipients as set out above. In some embodiments, compositions herein may be administered by the oral or nasal respiratory route for local or systemic effect.

In some embodiments, pharmaceutical compositions disclosed herein can be topical formulations. In some embodiments, topical formulations disclosed herein can be a cream, an ointment, a paste, a lotion, a gel, or any combination thereof. Some embodiments of the present disclosure provide pharmaceutical compositions that can include at least active agent (e.g., (e.g., any antibody or antigen-binding fragment thereof specifically binds an IL-4R, such as dupilumab) and one or more additional agents selected from gelling agents, protein stabilizers, surfactants, preservatives, antimicrobial agents, salts, or any combination thereof.

In some embodiments, pharmaceutical compositions or formulations disclosed herein can include at least one gelling agent having an average molecular weight (MW) of about 25,000 to about 1,500,000 Daltons. In other embodiments, the at least one gelling agent can be one or more celluloses or derivatives thereof. In accordance with these embodiments, the at least one gelling agent can include at least one cellulose or derivative thereof including, but not limited to, benzylcellulose (BC), ethylcellulose (EC), methylcellulose (MC), hydroxypropylmethylcellulose (HPMC), ethylhydroxyethylcellulose (EHEC), carboxymethylcellulose (CMC), hydroxypropylcellulose (HPC), hydroxyethylcellulose (HEC), hydroxyethylmethylcellulose (NEMC), sodium carboxymethylcellulose (CMCNa), cellulose acetate (CA), cellulose nitrate, cellulose sulphate, cellulose acetate phthalate (CAP), hydroxybutylcellulose, hydroxybutylmethylcellulose, carboxypropyl methylcellulose, hydroxypropyl methylcellulose (HPMC), cellulose acetate butyrate (CAB), microcrystalline cellulose (MCC), hydroxypropylmethylcellulose phthalate (HPMCP), cellulose acetate trimelitate (CAT), hydroxypropylmethylcellulose acetate succinate, or any combination thereof.

In some embodiments, compositions or formulations disclosed herein can include one or more protein stabilizers. In accordance with these embodiments, one or more protein stabilizers can include, but are not limited to, succinic anhydride, albumin, sialic acid, creatinine, glycine, histidine or other amino acid capable of stabilizing proteins, niacinamide, sodium acetyltryptophonate, zinc oxide, sucrose, glucose, lactose, trehalose, sorbitol, mannitol or other saccharide or disaccharide, glycerol, polyethylene glycols, sodium caprylate, sodium saccharin, or other suitable protein stabilizer or any combination thereof.

In some embodiments, compositions or formulations disclosed herein can include one or more surfactants. In accordance with these embodiments, one or more surfactants of use in compositions disclosed herein can include, but are not limited to, sodium lauryl sulfate, sodium decussate, Tween-20, Tween-60, Tween-80; triacetin, vitamin E TPGS, a phospholipid, a lecithin, a phosphatidyl choline, a phosphatidylethanolamine, a phosphatidylglycerol, sorbitan monooleate, polyoxyethylene sorbitan monooleate, a polysorbate, a polaxomer, bile salt, glyceryl monostearate, a copolymer of ethylene oxide and propylene oxide, polyoxyethylene hydrogenated castor oil, polyoxyethylene alkylether, octoxynol 10, octoxynol 40, or any combination thereof.

In some embodiments, compositions or formulations disclosed can include one or more preservatives that can be benzalkonium chloride, chlorobutanol, thimerosol, chloroxylenol, chlorhexidine, phenoxyethanol, benzyl alcohol, phenethyl alcohol, polyquaterniaum-1, diazolidinyl urea, iodopropynyl butylcarbamate, chloromethylisotiazolinone, methylisothiazolinone, vitamin E, a vitamin E derivative, vitamin E acetate, vitamin C, butylated hydroxytoluene, butylparaben, ethylparaben, methylparaben, propylparaben, isobutylparaben, phenoxyethanol, ethylparaben, propylparaben, utylparaben, or any combination thereof. In some embodiments, compositions herein can be free of preservatives.

### IV. Therapeutic Applications

In certain embodiments, the present disclosure provides methods for improving severity of hair loss in a subject having or suspected of having alopecia areata (AA) using one or more of the antibodies and/or antigen-binding fragments thereof that specifically binds IL-4R as disclosed herein. In some embodiments, methods herein for improving severity of hair loss in a subject having or suspected of having AA can include administration of an IL-4R antibody. In some examples, methods herein for improving severity of hair loss in a subject having or suspected of having AA can include administration of dupilumab. In some examples, methods herein for improving severity of hair loss in a subject having or suspected of having AA can include determining a baseline IgE serum level in the subject before administering one or more of the antibodies and/or antigen-binding fragments thereof that specifically binds IL-4R as disclosed herein.

In some embodiments, to practice the therapeutic methods described herein, an effective amount of an IL-4R antibody (e.g., dupilumab) described herein or a pharmaceutical composition comprising such may be administered to a subject who needs treatment via a suitable route (e.g., topical, systemic, subcutaneous, intravenous, or intranasal administration). In some embodiments, the IL-4R antibody (e.g., dupilumab) may be mixed with a pharmaceutically acceptable carrier to form a pharmaceutical composition (e.g., see disclosures herein) prior to administration, which is also within the scope of the present disclosure.

In some embodiments, a subject to be treated by any of the methods disclosed herein may be a mammal (e.g., a human patient or a non-human primate). In some embodiments, a subject may have, be suspected of having, or be at risk for AA. In some embodiments, a subject may have partial (patchy AA) to complete scalp hair loss (alopecia totalis [AT]) or to total scalp, face, and body hair loss (alopecia universalis [AU]). In some embodiments, a subject having AA can be identified by routine medical examination, e.g., laboratory tests, functional tests, and the like. In some embodiments, a subject that may have, be suspected of having, or be at risk for AA can have their IgE levels determined to assess for the presence of AA, the severity of AA, or both. In some embodiments, a subject that may have, be suspected of having, or be at risk for AA can have their IgE levels determined prior to being subjected to the methods disclosed herein. In some embodiments, a subject that may have, be suspected of having, or be at risk for AA can have their IgE levels determined during the administration of one or more of the methods disclosed herein.

In some embodiments, a subject to be treated by any of the methods disclosed herein can be identified as suitable for treatment by measuring the subject's total immunoglobulin E (IgE) serum levels. IgE is one of the 5 classes (isotypes) of antibodies. Like other immunoglobulins, IgE is produced by B cells and plasma cells. In contrast to other immunoglobulins, the circulating concentration of IgE in a healthy subject can be very low because B cells synthesize IgE at a very low rate and mast cells, basophils, and activated eosinophils bind up most of the circulating IgE. The normal concentration of IgE in a healthy subject can be about 0.05% of the IgG concentration. Total IgE serum levels can be measured using routine methods known in the art. In some embodiments, total IgE serum can be measured in a subject herein using an electrochemiluminescence immunoassay, such as, but not limited to, an ELISA (enzyme-linked immunosorbent assay).

In some embodiments, a subject that can be identified as suitable for treatment herein by measuring the subject's total IgE serum level can be a human subject. For a healthy, non-allergic human subject, a total IgE serum level can range from about 0 IU/ml to about 200 IU/ml. For a healthy, non-allergic adult human subject, a total IgE serum level can range from about 0 IU/ml to about 100 IU/ml wherein an “adult human subject” refers to a human subject that is about 16 years of age or older. In some embodiments, a human subject having, suspected of having, or at risk for AA can have a total IgE serum level higher than a healthy, non-allergic human subject. In some embodiments, a human adult subject having, suspected of having, or at risk for AA can have a total IgE serum level higher than a healthy, non-allergic adult human subject. In some embodiments, a human subject having, suspected of having, or at risk for AA who has a total IgE serum level equal to or greater than about 200 IU/ml can be identified as a subject suitable for being treated by one or more of the methods disclosed herein.

In some embodiments, a human subject having, suspected of having, or at risk for AA that has a total IgE serum level equal to or greater than about 200 IU/ml can be predicted to respond to any of the methods disclosed herein. In some embodiments, a human subject having, suspected of having, or at risk for AA that has a total IgE serum level equal to or greater than about 200 IU/ml can be predicted to respond to methods disclosed herein with at least 75% (e.g., 75%, 80%, 85%, 90%, 95%, 98%, 100%) accuracy. In some embodiments, a human subject having, suspected of having, or at risk for AA that has a total IgE serum level equal to or greater than about 200 IU/ml can be predicted to respond to methods of administering at least one treatment for alopecia areata. In some embodiments, a treatment for alopecia areata can be a steroid, an immunosuppressive agent, a potassium channel activator, a janus kinase (JAK) inhibitor, a Tyrosine Kinase (TYK) inhibitor, an interleukin-4 (IL-4) blocker, an interleukin-4 receptor (IL-4R) blocker, an interleukin 5 (IL-5) blocker, an interleukin 5 receptor (IL-5R) blocker, an interleukin 12/23 p 40 (IL-12/23) blocker, an interleukin 13 (IL-13) blocker, an interleukin 13 receptor (IL-13R) blocker, an interleukin 23 (IL-23) blocker, a type II helper T cell (Th2) cytokine inhibitor, a modulator of sphingosine-1-phosphate (S1P), a modulator of natural killer group 2 member D (NKG2D), a modulator of thymic stromal lymphopoietin (TSLP), or any combination thereof. In some embodiments, a treatment for alopecia areata can be cyclosporine A, OX40, OX40 ligand (OX40L), abrocitinib, baricitinib, ritlecitinib, CTP 543, ustekinumab, tralokinumab, lebrikizumab, nemolizumab, upadacitinib, tofacitinib, ruxolitinib, oclacitinib, peficitinib, fedratinib, filgotinib, cerdulatinib, gandotinib, lestaurtinib, momelotinib, pacritinib, BMS-986165, dupilumab, pitrakinra, CBP 201, AK 120, pascolizumab, or any combination thereof. One of skill in the art would readily recognize other useful treatments of alopecia areata that are not mentioned above, which may be employed in the operation of the present disclosure.

In some embodiments, a human subject having, suspected of having, or at risk for AA that has a total IgE serum level equal to or greater than about 200 IU/ml can be predicted to respond to methods of administering at least one treatment for alopecia areata as disclosed herein with about 80% to about 85% (e.g., 80%, 81%, 82%, 83%, 84%, 85%) accuracy. In some embodiments, a human subject having, suspected of having, or at risk for AA that has a total IgE serum level equal to or greater than about 200 IU/ml can be predicted to respond to methods of administering at least one treatment for alopecia areata for about 24 weeks as disclosed herein with about 80% to about 85% (e.g., 80%, 81%, 82%, 83%, 84%, 85%) accuracy. In some embodiments, a human subject having, suspected of having, or at risk for AA that has a total IgE serum level equal to or greater than about 200 IU/ml can be predicted to respond to methods of administering at least one treatment for alopecia areata for about 48 weeks as disclosed herein with about 80% to about 85% (e.g. 80%, 81%, 82%, 83%, 84%, 85%) accuracy. In some embodiments, a human subject having, suspected of having, or at risk for AA that has a total IgE serum level equal to or greater than about 200 IU/ml can be predicted to respond to methods of administering at least one treatment for alopecia areata for longer than about 48 weeks as disclosed herein with about 80% to about 85% (e.g. 80%, 81%, 82%, 83%, 84%, 85%) accuracy. In some embodiments, a human subject having, suspected of having, or at risk for AA that has a total IgE serum level equal to or greater than about 200 IU/ml can be predicted to respond to methods of administering at least one treatment for alopecia areata for about 72 weeks as disclosed herein with about 80% to about 85% (e.g. 80%, 81%, 82%, 83%, 84%, 85%) accuracy. In some embodiments, a human subject having, suspected of having, or at risk for AA that has a total IgE serum level equal to or greater than about 200 IU/ml can be predicted to respond to methods of administering at least one treatment for alopecia areata for longer than about 72 weeks as disclosed herein with about 80% to about 85% (e.g. 80%, 81%, 82%, 83%, 84%, 85%) accuracy. In some embodiments, a human subject having, suspected of having, or at risk for AA that has a total IgE serum level equal to or greater than about 200 IU/ml can be predicted to respond to methods of chronically administering at least one treatment for alopecia areata as disclosed herein with about 80% to about 85% (e.g. 80%, 81%, 82%, 83%, 84%, 85%) accuracy.

In some embodiments, a human subject having, suspected of having, or at risk for AA that has a total IgE serum level equal to or greater than about 200 IU/ml can be predicted to respond to methods of administering dupilumab as disclosed herein with about 80% to about 85% (e.g., 80%, 81%, 82%, 83%, 84%, 85%) accuracy. In some embodiments, a human subject having, suspected of having, or at risk for AA that has a total IgE serum level equal to or greater than about 200 IU/ml can be predicted to respond to methods of administering dupilumab for about 24 weeks as disclosed herein with about 80% to about 85% (e.g., 80%, 81% 82%, 83%, 84%, 85%) accuracy. In some embodiments, a human subject having, suspected of having, or at risk for AA that has a total IgE serum level equal to or greater than about 200 IU/ml can be predicted to respond to methods of administering dupilumab for about 48 weeks as disclosed herein with about 80% to about 85% (e.g. 80%, 81%, 82%, 83%, 84%, 85%) accuracy. In some embodiments, a human subject having, suspected of having, or at risk for AA that has a total IgE serum level equal to or greater than about 200 IU/ml can be predicted to respond to methods of administering dupilumab for longer than about 48 weeks as disclosed herein with about 80% to about 85% (e.g. 80%, 81%, 82%, 83%, 84%, 85%) accuracy. In some embodiments, a human subject having, suspected of having, or at risk for AA that has a total IgE serum level equal to or greater than about 200 IU/ml can be predicted to respond to methods of administering dupilumab for about 72 weeks as disclosed herein with about 80% to about 85% (e.g. 80%, 81%, 82%, 83%, 84%, 85%) accuracy. In some embodiments, a human subject having, suspected of having, or at risk for AA that has a total IgE serum level equal to or greater than about 200 IU/ml can be predicted to respond to methods of administering dupilumab for longer than about 72 weeks as disclosed herein with about 80% to about 85% (e.g. 80%, 81%, 82%, 83%, 84%, 85%) accuracy. In some embodiments, a human subject having, suspected of having, or at risk for AA that has a total IgE serum level equal to or greater than about 200 IU/ml can be predicted to respond to methods of chronically administering dupilumab as disclosed herein with about 80% to about 85% (e.g. 80%, 81%, 82%, 83%, 84%, 85%) accuracy.

In some embodiments, a receiver operating characteristic (ROC) curve analysis of IgE serum levels can be used to determine a threshold that would differentiate a human subject having, suspected of having, or at risk for AA that would respond to disclosed methods of administering dupilumab from a human subject having, suspected of having, or at risk for AA that would not respond to the methods of administering dupilumab as disclosed herein. ROC represents a probability curve and AUC (area under the curve) represents a measure of separability (i.e., how much a model is capable of distinguishing between classes). It is understood that the higher the AUC, the better the model is at distinguishing between the human subjects who will respond to dupilumab treatment of AA and the human subjects who will not respond to dupilumab treatment of AA. In some embodiments, a ROC curve analysis of total serum IgE in a human subject for responsiveness to dupilumab treatment for AA as disclosed herein can have an AUC of at least about 0.75 (e.g., 0.75, 0.80, 0.85, 0.90, 0.95, 0.99, 1.0). In some embodiments, a ROC curve analysis of total serum IgE in a human subject for responsiveness to dupilumab treatment for AA as disclosed herein can have an AUC of about 0.80 to about 0.85 (e.g., 0.80, 0.81, 0.82, 0.83, 0.84, 0.85).

In some embodiments, determining a concentration of a total IgE in a serum sample collected from a subject to be treated with any of the compositions herein can be used as a method of determining the likelihood of improving severity of hair loss in the subject to be treated. In some embodiments, methods of determining the likelihood of improving severity of hair loss in the subject to be treated with a composition herein can include measuring a total IgE in a serum sample from the subject to be treated and comparing the concentration of total IgE to a control. In some embodiments, a control sample can be obtained by measuring a total IgE in a serum sample from a population of normal healthy control subjects. In some embodiments, a control sample obtained by measuring total IgE in serum samples from a population of normal healthy control subjects can have a total IgE of less than about 200 In some embodiments, a subject to be treated with a composition herein can have a high likelihood of improving severity of hair loss when the total IgE in the serum sample collected from the subject is equal to or higher than total IgE measured in a control sample. In some embodiments, a subject to be treated with a composition herein can have a high likelihood of improving severity of hair loss when the total IgE in the serum sample collected from the subject is equal to or higher than 200 IU/ml.

In some embodiments, the odds ratio (OR) of response in subjects having a high total serum IgE treated with a composition herein compared to subjects having a normal total serum IgE treated with a composition herein can be determined. In some embodiments, the OR of response in subjects having a total serum IgE equal to or greater than 200 IU/ml treated with a composition herein compared to subjects having a total serum IgE less than 200 IU/ml treated with a composition herein can be determined. When calculating OR, the response can be an increase in Severity of Alopecia Tool (SALT) score over baseline wherein baseline is the SALT score taken before administration of a composition disclosed herein. In some embodiments, OR can be about 8 where the response is at least about a 30% improvement in SALT score after treatment with a composition herein compared to baseline. In some embodiments, OR can be about 8 where the response is at least about a 30% improvement in SALT score after an about 1 week to about 72 weeks treatment with a composition herein compared to baseline.

In some embodiments, a subject to be treated by the methods described herein may be a human patient who has undergone or is currently subjected to a therapy for treating AA. In some embodiments, the prior AA therapy may be complete. In some embodiments, the prior AA therapy may still be on-going. In some embodiments, the prior AA therapy can be administration of another active agent. In some embodiments, other active agents that can be used for treating AA prior to or during the course of the methods disclosed herein can be corticosteroids, immunosuppressive agents (e.g., cyclosporine A), potassium channel activators, janus kinase (JAK) inhibitors, Tyrosine Kinase (TYK) inhibitors, interleukin 5 (IL-5) blockers, interleukin 5 receptor (IL-5R) blockers, interleukin 12/23 p 40 (IL-12/23) blockers, interleukin 13 (IL-13) blockers, interleukin 13 receptor (IL-13R) blockers, interleukin 23 (IL-23) blockers, Th2 cytokine inhibitors (e.g., including but not limited to OX40 (also known as CD134 or TNFRSF4), OX40 ligand (OX40L), modulators of sphingosine-1-phosphate (51P), modulators of NKG2D, and modulators of thymic stromal lymphopoietin (TSLP)), or any combination thereof. In some embodiments, other active agents that can be used for treating AA prior to or during the course of the methods disclosed herein can be an IL-4 blocker, and IL-4R blocker, or any combination thereof that has not been disclosed in detail herein.

In some embodiments, the subject may be a human adult patient (e.g., an adult patient having AA). In some embodiments, the subject may be a human child patient (e.g., a child patient having AA). Such a child patient may be younger than 18 years. In some embodiments, a child patient to be treated by the methods disclosed herein may have an age younger than 12, for example, younger than 10, 8, or 6.

In some embodiments, a pharmaceutical composition comprising any of the IL-4R antibodies (e.g., dupilumab) disclosed herein may be administered by any administration route known in the art, such as but not limited to parenteral administration, oral administration, buccal administration, sublingual administration, topical administration, or inhalation, in the form of a pharmaceutical formulation comprising the active ingredient, optionally in the form of a non-toxic organic, or inorganic, acid, or base, addition salt, in a pharmaceutically acceptable dosage form. In some embodiments, the administration route may be subcutaneous injection and the formulation is formulated for subcutaneous administration.

“An effective amount” as used herein refers to the amount of each active agent (here the one or more IL-4R antibodies (e.g., dupilumab)) required to confer therapeutic effect on the subject, either alone or in combination with one or more other active agents. Effective amounts vary, as recognized by those skilled in the art, depending on route of administration, excipient usage, and co-usage with other active agents. For example, an “effective amount” of an IL-4R antibody (e.g., dupilumab) may be the amount of the antibody that alone, or together with further doses, produces one or more desired responses, e.g., improve severity of hair loss, improve quality of life, and/or improve severity of Alopecia Tool (SALT) score in a subject having AA. In some embodiments, an effective amount may slow the progression of the disease temporarily and/or may halt the progression of the disease permanently. This can be monitored by routine methods or can be monitored according to diagnostic methods of the present disclosure. In some embodiments, a desired response to treatment of the disease or condition also can be delaying the onset of the disease or condition. In some embodiments, an effective amount is the amount of an IL-4R antibody (e.g., dupilumab) that regrows hair in a subject having AA.

In some embodiments, an effective amount can be the amount of an IL-4R antibody (e.g., dupilumab) that can reduce an Alopecia Areata Symptom Impact Scale (AASIS) score by at least about 5%. In some embodiments, an effective amount can be the amount of an IL-4R antibody (e.g., dupilumab) that can reduce an AASIS score by at about 5% to about 40% (i.e., 5%, 10%, 15%, 20%, 25%, 30%, 35%, 40%).

In some embodiments s, an effective amount can be the amount of an IL-4R antibody (e.g., dupilumab) that can reduce an AASIS score by at least about 5% after 24 weeks of administration according to methods herein. In some embodiments, an effective amount can be the amount of an IL-4R antibody (e.g., dupilumab) that can reduce an AASIS score by at least about 5% after 48 weeks of administration according to methods herein.

In some embodiments, an effective amount can be the amount of an IL-4R antibody (e.g., dupilumab) that can improve a Quality of Life (QoL) score by at least about 5%. In some embodiments, an effective amount can be the amount of an IL-4R antibody (i.e., dupilumab) that can improve a QoL score by at about 5% to about 40% (i.e., 5%, 10%, 15%, 20%, 25%, 30%, 35%, 40%). In some embodiments, an effective amount can be the amount of an IL-4R antibody (e.g., dupilumab) that can improve a QoL score by at least about 5% after 24 weeks of administration according to methods herein. In some embodiments, an effective amount can be the amount of an IL-4R antibody (e.g., dupilumab) that can improve a QoL score by at least about 5% after 48 weeks of administration according to methods herein.

In some embodiments, an effective amount can be the amount of an IL-4R antibody (e.g., dupilumab) that can improve an Eczema Area and Severity Index (EASI) score by at least about 5%. In some embodiments, an effective amount can be the amount of an IL-4R antibody (e.g., dupilumab) that can improve a EASI score by at about 5% to about 40% (i.e., 5%, 10%, 15%, 20%, 25%, 30%, 35%, 40%). In some embodiments, an effective amount can be the amount of an IL-4R antibody (e.g., dupilumab) that can improve an EASI score by at least about 5% after 24 weeks of administration according to methods herein. In some embodiments, an effective amount can be the amount of an IL-4R antibody (e.g., dupilumab) that can improve an EASI score by at least about 5% after 48 weeks of administration according to methods herein.

Such amounts will depend on the condition being treated, the severity of the condition, the individual subject's parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons. The exact dosage and schedule may be determined by a physician.

In some embodiments, dosages for an antibody as described herein (e.g., dupilumab) may be determined empirically in subjects who have been given one or more administration(s) of the antibody. In some embodiments, subjects may be given incremental dosages of the antibody. To assess efficacy of the antibody, an indicator of the disease/disorder can be followed.

In some embodiments, for an adult patient of normal weight, doses of an anti-IL-4R antibody as described herein (e.g., dupilumab) ranging from about 100 to 600 mg/kg may be administered. In some embodiments, an initial dosage of the anti-IL-4R antibody (e.g., dupilumab) described herein can be about 300 mg/kg for an adult human patient. In some embodiments, an initial dosage can be immediately followed by a secondary dosage of the anti-IL-4R antibody (e.g., dupilumab) described herein, wherein the secondary dosage can be about 300 mg/kg to about 600 mg/kg for an adult human patient. In some embodiments, secondary dosages of an anti-IL-4R antibody as described herein (e.g., dupilumab) can be about 300 mg/kg to about 600 mg/kg for an adult human patient. In some embodiments, an initial dosage of the anti-IL-4R antibody (e.g., dupilumab) described herein can be about 100 mg/kg to 600 mg for a child human patient. In some embodiments, the initial dosage can be immediately followed by a secondary dosage of the anti-IL-4R antibody (e.g., dupilumab) described herein, wherein the secondary dosage can be about 100 mg/kg to about 600 mg/kg for a child human patient. In some embodiments, secondary dosages can be about 100 mg/kg to about 600 mg/kg for a child human patient.

In some embodiments, the particular dosage regimen, i.e., dose, timing and repetition, can depend on the particular individual and that individual's medical history, as well as the properties of the individual agents (such as the half-life of the agent, and other considerations well known in the art). In accordance with some embodiments herein, the progress of this therapy can be easily monitored by conventional techniques and assays. In some embodiments, the dosing regimen (including the antibody herein used) can vary over time.

In some embodiments, a secondary dosage of the anti-IL-4R antibody (e.g., dupilumab) described herein can be administered about 5 days to about 10 days after the initial dose of the anti-IL-4R antibody (e.g., dupilumab) described herein. In some embodiments, a secondary dosage of the anti-IL-4R antibody (e.g., dupilumab) described herein can be administered 7 days after the initial dose of the anti-IL-4R antibody (e.g., dupilumab) described herein. In some embodiments, secondary doses following the first secondary dose can be administered in a fixed range of times apart as needed. In some embodiments, a fixed range of times apart for administration of subsequent secondary doses can be about 5 days to about 10 days. In some embodiments, a fixed range of times apart for administration of subsequent secondary doses can be 7 days.

In some embodiments, dosing of the anti-IL-4R antibody (e.g., dupilumab) as described herein can be determined based on baseline IgE levels. In some embodiments, dosing of the anti-IL-4R antibody (e.g., dupilumab) as described herein can be determined based on body weight. In some embodiments, dosing of the anti-IL-4R antibody (e.g., dupilumab) as described herein can be determined based on a matrix of baseline IgE levels and body weight.

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the agonist, and/or the discretion of the attending physician. In some embodiments, a clinician can administer an antibody until a dosage is reached that achieves the desired result. In some embodiments, the desired result can be an increase in hair growth. Methods of determining whether a dosage resulted in the desired result would be evident to one of skill in the art. In some embodiments, administration of one or more antibodies disclosed herein (e.g., dupilumab) can be continuous or intermittent, depending, for example, upon the recipient's physiological condition, whether the purpose of the administration is therapeutic or prophylactic, and/or other factors known to skilled practitioners. In some embodiments, administration of one or more antibodies disclosed herein (e.g., dupilumab) may be continuous over a preselected period of time or may be in a series of spaced doses, e.g., either before, during, or after developing a target disease or disorder.

As used herein, the term “treating” refers to the application or administration of a composition herein including one or more active agents (e.g., dupilumab) to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

In some embodiments, administration of a composition disclosed herein to a subject in need thereof can improve the Severity of Alopecia Tool (SALT) score in the treated subject. SALT is a quantitative assessment of alopecia areata (AA) severity based on the scalp hair loss. Absolute changes in the SALT score compared to baseline or the percent change from baseline can be used to track response to treatment with any of the compositions administered herein. As used herein, baseline is the SALT score obtained prior to administering a composition herein to a subject. For recording purposes, the percent change from baseline can be noted as a subscript of the SALT score (e.g., 30% improvement=SALT30). In some embodiments, a subject administered a composition herein can have a SALT score of SALT30 after at least about 2 weeks of treatment. In some embodiments, a subject administered a composition herein can have a SALT score of SALT30 after about 2 weeks to about 72 weeks of treatment. In some embodiments, a subject administered a composition herein can have a SALT score of SALT30 after longer than about 72 weeks of treatment. In some embodiments, a subject administered a composition herein can have a SALT score of SALT30 after about 8 weeks of treatment. In some embodiments, a subject administered a composition herein can have a SALT score of SALT50 after at least about 2 weeks of treatment. In some embodiments, a subject administered a composition herein can have a SALT score of SALT50 after about 2 weeks to about 72 weeks of treatment. In some embodiments, a subject administered a composition herein can have a SALT score of SALT50 after longer than about 72 weeks of treatment. In some embodiments, a subject administered a composition herein can have a SALT score of SALT50 after about 12 weeks of treatment. In some embodiments, a subject administered a composition herein can have a SALT score of SALT75 after at least about 2 weeks of treatment. In some embodiments, a subject administered a composition herein can have a SALT score of SALT75 after about 2 weeks to about 72 weeks of treatment. In some embodiments, a subject administered a composition herein can have a SALT score of SALT75 after longer than about 72 weeks of treatment. In some embodiments, a subject administered a composition herein can have a SALT score of SALT75 after about 20 weeks of treatment. In some embodiments, a subject having a total serum IgE equal to or higher than 200 IU/ml can have a higher SALT score after treatment with a composition herein compared to a subject having a total serum IgE less than 200 IU/ml after treatment with a composition herein. In some embodiments, a subject having a total serum IgE equal to or higher than 200 IU/ml can have an increased response from SALT30 to SALT50 after treatment with a composition herein compared to a subject having a total serum IgE less than 200 IU/ml after treatment with a composition herein.

“Development” or “progression” of a disease means initial manifestations and/or ensuing progression of the disease. Development of the disease can be detectable and assessed using standard clinical techniques as well known in the art. However, development may also refer to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. In some embodiments, compositions herein can be administered via conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In some embodiments, compositions herein can be administered to the subject via injectable depot routes of administration such as using 1-, 3-, or 6-month depot injectable or biodegradable materials and methods. In some embodiments, compositions herein can be administered intraocularly or intravitreally.

In some embodiments, compositions herein can be injectable compositions. Injectable compositions herein may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

The particular dosage regimen, i.e., dose, timing and repetition, used in the methods described herein may depend on the particular subject and that subject's medical history.

In some embodiments, more than one antibody, or a combination of an antibody and another suitable therapeutic agent, may be administered to a subject in need of the treatment. In some embodiments, an antibody herein can also be used in conjunction with other agents that serve to enhance and/or complement the effectiveness of the agents. Treatment efficacy for a target disease/disorder can be assessed by methods well-known in the art.

### V. Kits for Use in Treatment of AA

The present disclosure also provides kits for use in treating or alleviating a target disease, such as AA as described herein. Such kits can include one or more containers comprising an anti-IL-4R antibody, e.g., dupilumab. In some instances, the anti-IL-4R antibody may be co-used with a second therapeutic agent.

In some embodiments, kits herein can comprise instructions for use in accordance with any of the methods described herein. The included instructions can comprise a description of administration of the anti-IL-4R antibody (e.g., dupilumab), and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein.

In some embodiments, a kit disclosed herein can include components necessary to determine a subjects IgE level and include one or more containers comprising an anti-IL-4R antibody, e.g., dupilumab. In some embodiments, a kit disclosed herein can include an electrochemiluminescence immunoassay suitable for measuring serum IgE levels. In some embodiments, a kit disclosed herein can include instructions, buffers, and the like needed to perform an electrochemiluminescence immunoassay for measuring serum IgE levels.

In some embodiments, the instructions provided in a kit herein may comprise a description of administering an antibody to an individual at risk of the target disease. The instructions relating to the use of an anti-IL-4R antibody may generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert may indicate that the composition is used for treating, delaying the onset and/or alleviating the disease, such as an immune disorder (e.g., alopecia areata (AA)). Instructions may be provided for practicing any of the methods described herein.

The kits of the present disclosure can be in suitable packaging. Suitable packaging includes, but is not limited to, vials, bottles, jars, flexible packaging (e.g., sealed Mylar or plastic bags), and the like. Also contemplated herein are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-IL-4R antibody (e.g., dupilumab) as those described herein.

Kits may optionally provide additional components such as buffers and interpretive information. In some embodiments, a kit can comprise a container and a label or package insert(s) on or associated with the container. In some embodiments, the invention provides articles of manufacture comprising contents of the kits described above.

### VI. General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1

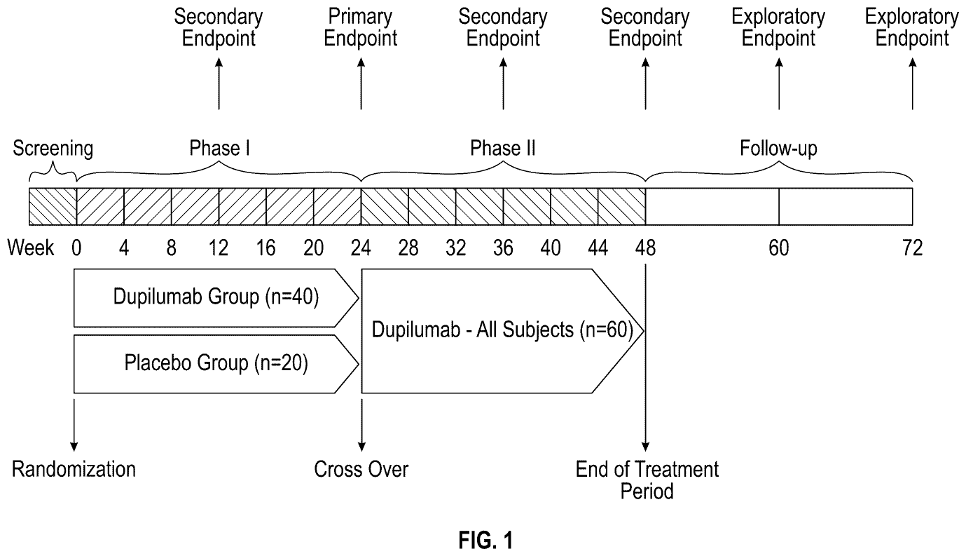

The purpose of this study was to assess whether dupilumab can be a helpful treatment for alopecia areata. The study was a phase 2a, randomized, double-blind, multicenter study with an initial 24-week evaluation period (primary endpoint), followed by another 24-week open-label phase in which all participants were treated with dupilumab up to week 48 (secondary endpoint), with a follow-up to week 72 (FIG. 1). Patients were randomized 2:1 to receive weekly dupilumab (300 mg) or matching placebo. The primary efficacy endpoint was change from baseline in the Severity of Alopecia Tool (SALT) score at week 24. The SALT score quantifies scalp hair loss, and ranges from 0 (no hair loss) to 100 (complete hair loss).

Key secondary outcomes included a change in SALT from week 48 to week 24 and to baseline and the proportion of patients achieving 30%/50%/75% SALT score improvement (SALT_{30/50/75}) at week 24 and 48. Other outcomes included change in the Alopecia Areata Symptom Impact Scale (AASIS) and Alopecia Areata Quality of Life questionnaires (AA-QoL), the proportion of patients with Alopecia Areata Physician's Global Assessment (AA-PGA) improvement, change in eyelash and eyebrow scores and in Eczema Area and Severity Index (EASI), and safety assessments. For AA-PGA scoring: no regrowth was scored as 0; <25% regrowth was scored as 1; 25-49% regrowth was scored as 2; 50-74% regrowth was scored as 3; 75-99% regrowth was scored as 4; and 100% regrowth was scored as 5. Safety parameters included treatment-emergent adverse events (AEs) throughout the study and clinical laboratory abnormalities. Safety labs and blood markers potentially related with AA/atopy (i.e, lactate dehydrogenase [LDH], C-reactive protein [CRP], percentage of eosinophils, and total serum IgE) were evaluated at baseline and weeks 12, 24, 36 and 48.

The sample size was based on the primary efficacy outcome, i.e., a change in SALT score at week 24 in drug compared to placebo. For patients with similar disease severity, a change in SALT of less than 5 units was observed in placebo. Assuming that at week 24 dupilumab will induce a change in SALT of at least 25 with a standard deviation of 10, a total sample size of 54 patients randomized 2:1 dupilumab to placebo will provide 97% power to detect differences compared to placebo, based on a two-sided Student's t-test with a type I error α=0.05. Assuming a 10% dropout rate, the planned sample size was a total of 60 patients.

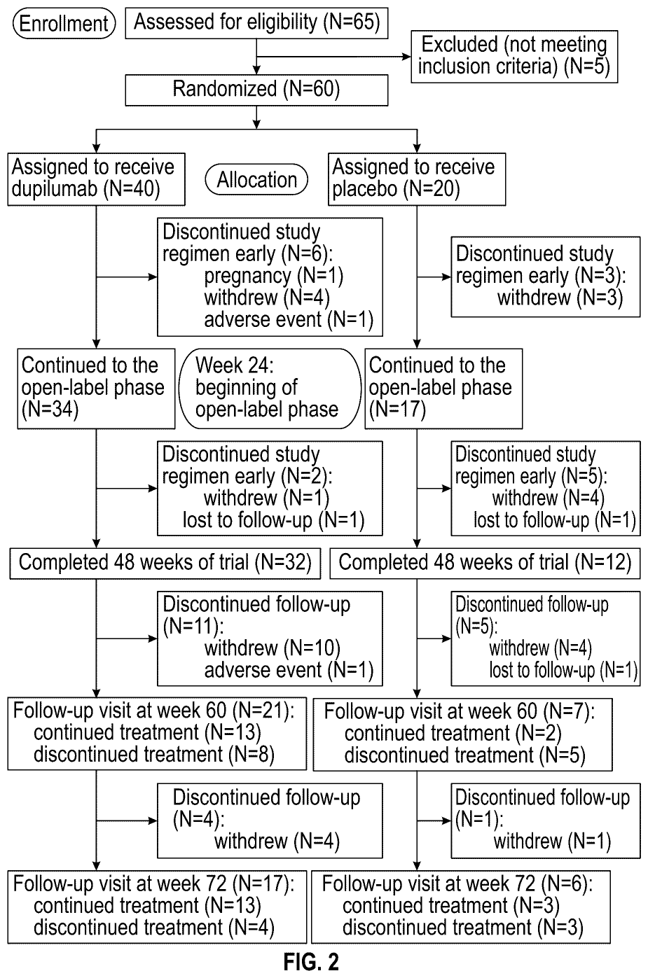

The primary outcome was analyzed by a linear mixed-effect model repeated measures with treatment and time point interaction as a fixed effect and a random effect for each subject. For continuous secondary outcomes we used a similar approach by linear mixed-effect models, stratified by variables such as IgE levels and atopy. Categorical outcomes were evaluated as two-sample proportions by Fisher's exact test; patients with missing values were considered non-responders. Confidence intervals have not been adjusted for multiplicity and cannot be used to infer definitive treatment effects for secondary efficacy endpoints. Correlation analysis was conducted using Pearson correlation coefficient, and P-values were adjusted by false discovery rate. To evaluate the utility of IgE as a possible predictor of dupilumab response in AA (for a SALT₅₀ response at week 24), we used the receiver operating characteristic area under the curve (AUC). Trial findings are described in accordance with CONSORT guidelines (FIG. 2).

The inclusion and exclusion criteria for the study were as follows: Inclusion Criteria:
Male or female subjects who are at least 18 years old at the time of informed consent.
Subject was able to understand and voluntarily sign an informed consent document prior to participation
Subject was able to adhere to the study visit schedule and other protocol requirements.
Females of childbearing potential (FCBP) must have a negative pregnancy test at Screening and Baseline. While on investigational product and for at least 28 days after taking the last dose of investigational product (IP), FCBP who engage in activity in which conception was possible must use one of the approved contraceptive options described below:
   Option 1: Any one of the following highly effective methods: hormonal contraception (oral, injection, implant, transdermal patch, vaginal ring); intrauterine device (IUD); tubal ligation; or partner's vasectomy; OR
   Option 2: Male or female condom (latex condom or non-latex condom NOT made out of natural [animal] membrane [for example, polyurethane]); PLUS one additional barrier method: (a) diaphragm with spermicide; (b) cervical cap with spermicide; or (c) contraceptive sponge with spermicide.
If subject was a female of non-childbearing potential, she must have documented history of infertility, be in a menopausal state for one year, or had a hysterectomy, bilateral tubal ligation, or bilateral oophorectomy.
Subject had a history of at least 6 months of moderate to severe AA 30% scalp involvement) as measured using the SALT score; OR subject has 95% loss of scalp hair for enrollment as AA totalis (AT) or universalis (AU) subtypes.
   AT and AU were limited to 50% of the total number of subjects enrolled.
   One-third of subjects must have active AD skin or a concomitant history of AD at the time of the Screening and Baseline visits.
Subject had a negative Tuberculin purified protein derivative (PPD) or QuantiFERON TB-Gold test (QFT) prior to baseline. Subjects with a positive or indeterminable PPD or QFT result must have had a documented negative workup for tuberculosis and/or completed standard tuberculosis therapy.
Subjects must have met the following laboratory criteria:
   White blood cell count≥3000/mm3 (≥3.0×109/L) and <14,000/mm3 (≤14×109/L).
   Platelet count ≥100,000/μL (≥100×109/L).
   Serum creatinine ≤1.5 mg/dL (≤132.6 μmol/L).
   AST (SGOT) and ALT (SGPT)≤2×upper limit of normal (ULN). If the initial test showed ALT or AST>2 times the ULN, one repeat test was allowed during the Screening Phase.
   Total bilirubin ≤2 mg/dL (34 μmol/L). If the initial test showed total bilirubin >2 mg/dL (34 μmol/L), one repeat test was allowed during the Screening Phase.
   Hemoglobin 10 g/dL 6.2 mmol/L).
Subject was judged to be in otherwise good overall health following a detailed medical and medication history, physical examination, and laboratory testing.

### Exclusion Criteria:

Subject was pregnant or breastfeeding.
Subject's cause of hair loss was indeterminable and/or they have concomitant causes of alopecia, such traction, cicatricial, pregnancy-related, drug-induced, telogen effluvium, or advanced androgenetic alopecia (i.e. Ludwig Type III or Norwood-Hamilton Stage≥V).
Subject had a history of AA with no evidence of hair regrowth for ≥10 years since their last episode of hair loss.
Subject had an active bacterial, viral, or helminth parasitic infections; OR a history of ongoing, recurrent severe infections requiring systemic antibiotics Subject with a known or suspected underlying immunodeficiency or immune-compromised state as determined by the investigator.
Subject had a concurrent or recent history of severe, progressive, or uncontrolled renal, hepatic, hematological, intestinal, metabolic, endocrine, pulmonary, cardiovascular, or neurological disease.
Active hepatitis B, hepatitis C, human immunodeficiency virus (HIV), or positive HIV serology at the time of screening for subjects was determined by the investigators to be at high-risk for this disease.
Subject had a suspected or active lymphoproliferative disorder or malignancy; OR a history of malignancy within 5 years before the Baseline assessment, except for completely treated in situ non-melanoma skin and cervical cancers without evidence of metastasis.
Subject had received a live attenuated vaccine ≤30 days prior to study randomization.
Subject had any uncertain or clinically significant laboratory abnormalities that may affect interpretation of study data or endpoints.
Subject had any other medical or psychological condition that, in the opinion of the investigator, presented additional unreasonable risks as a result of their participation in the study and/or interfere with clinic visits and necessary study assessments.
History of adverse systemic or allergic reactions to any component of the study drug.
Severe, untreated asthma or a history of life-threatening asthma exacerbations while on appropriate anti-asthmatic mediations.
Use of systemic immunosuppressive medications, including, but not limited to, cyclosporine, systemic or intralesional corticosteroids, mycophenolate mofetil, azathioprine, methotrexate, tacrolimus, or ultraviolet (UV) phototherapy with/without Psoralen Ultraviolet A (PUVA) therapy within 4 weeks prior to randomization.
Use of an oral JAK inhibitor (tofacitinib, ruxolitinib) within 12 weeks prior to the Baseline visit.
Subject had used topical corticosteroids, and/or tacrolimus, and/or pimecrolimus within 1 week before the Baseline visit.
Subject had been previously treated with dupilumab.
Subject was currently using or planned to use anti-retroviral therapy at any time during the study.

An initial 24-week evaluation period (primary endpoint) in which patients were randomized to receive weekly, subcutaneous dupilumab or matching placebo (300 mg) was followed by another 24-week open-label phase in which all participants were treated with weekly dupilumab up to week 48 (secondary endpoint). Patients self-administrated dupilumab/placebo at their home after given instructions and guidance from the research team on baseline visit. From baseline visit to week 48 visit, participants were evaluated every four weeks. After week 48, follow-up continued at weeks 60 and 72.

During the study, the following measures were assessed:
Change in SALT Score [Time Frame: Baseline and 24 weeks]
Percent change in SALT score at Weeks 24 compared to Baseline. SALT−Scalp was divided into four areas: vertex (40% of scalp surface area), right profile (18% of scalp surface area), left profile (18% of scalp surface area), and posterior scalp (24% of scalp surface area). Percentage of hair loss in these areas was multiplied by percent surface area of the scalp in that area. SALT score was the sum of percentage of hair loss in all areas.
Percent change in SALT [Time Frame: Baseline and 48 weeks]
Percent change in SALT score at Weeks 48 compared to Baseline. SALT−Scalp was divided into four areas: vertex (40% of scalp surface area), right profile (18% of scalp surface area), left profile (18% of scalp surface area), and posterior scalp (24% of scalp surface area). Percentage of hair loss in these areas was multiplied by percent surface area of the scalp in that area. SALT score was the sum of percentage of hair loss in all areas.
Change in SALT75 [Time Frame: Baseline and 72 weeks]
Proportion of patients with SALT-75 (> or equal to 75% improvement in SALT score) at Weeks 72 compared to Baseline. SALT−Scalp divided was into four areas: vertex (40% of scalp surface area), right profile (18% of scalp surface area), left profile (18% of scalp surface area), and posterior scalp (24% of scalp surface area). Percentage of hair loss in these areas was multiplied by percent surface area of the scalp in that area. SALT score was the sum of percentage of hair loss in all areas.
Change in SALT50 [Time Frame: Baseline and 72 weeks]
Proportion of patients with SALT-50 (> or equal to 50% improvement in SALT score) at Weeks 72 compared to Baseline. SALT−Scalp was divided into four areas: vertex (40% of scalp surface area), right profile (18% of scalp surface area), left profile (18% of scalp surface area), and posterior scalp (24% of scalp surface area). Percentage of hair loss in these areas was multiplied by percent surface area of the scalp in that area. SALT score was the sum of percentage of hair loss in all areas.
Change in Alopecia Areata Symptom Impact Scale (AASIS) [Time Frame: Baseline and 24 weeks]
Change in AASIS at Weeks 24 compared to Baseline. AASIS was a 13-item instrument, each item scored from 0 to 10 where higher scores corresponded to worse symptom impact, full range from 0 to 130.
Change in Alopecia Areata Symptom Impact Scale (AASIS) [Time Frame: Baseline and 48 weeks]
Change in AASIS at Weeks 48 compared to Baseline. AASIS was a 13-item instrument, each item scored from 0 to 10 where higher scores corresponded to worse symptom impact, full range from 0 to 130.
Percent change in Alopecia Areata Quality of Life questionnaire [Time Frame: Baseline and 24 weeks]
Percent change in Alopecia Areata Quality of Life (AAQoL) at Weeks 24 compared to baseline. AAQoL was a 21-item instrument scored from 0 (poor) to 100 (good).
Percent change in AAQoL [Time Frame: Baseline and 48 weeks]
Percent change in AAQoL at Weeks 48 compared to baseline. AAQoL was a 21-item instrument, each time scored from 0 to 4. The scores were adjusted to produce a full range from 0 to 100 where higher scores referred to worse QoL.
Change in Salt subclass score [Time Frame: Baseline and 24 weeks]
Semi-quantitative score using SALT subclasses (0=no hair loss; 1=<25% hair loss; 2=25-49% hair loss; 3=50-75% hair loss; 4=75-99% hair loss; 5=100% hair loss) at weeks 24 compared to baseline
Change in Salt subclass score [Time Frame: Baseline and 48 weeks]
Semi-quantitative score using SALT subclasses (0=no hair loss; 1=<25% hair loss; 2=25-49% hair loss; 3=50-75% hair loss; 4=75-99% hair loss; 5=100% hair loss) at weeks 48 compared to baseline
Investigator's Global Assessment (IGA) [Time Frame: Week 48]
The aaPGA was used to assess the clinical response to treatment was based on a 6-point scale ranging from 0 (no regrowth) to 5 (100% regrowth).
Change in the Eyelash/Eyebrow Assessment score [Time Frame: Baseline and 48 weeks]
The Eyelash/Eyebrow Assessment score was based on a 5-point scale, ranging from 0 (none) to 4 (very prominent eyelashes/eyebrows)
Change in Eczema Area and Severity Index (EASI90) [Time Frame: Baseline and 48 weeks]
Proportion of patients with EASI90 (> of equal to 90% reduction from baseline EASI score) at 48 weeks compared to baseline. The EASI was used to assess the severity and extent of AD. Four AD disease characteristics were assessed (0=absent to 3=severe). Area of AD involvement was assessed as a percentage by involved body area of the head, trunk, arms, and legs, and converted to a score of 0 to 6.
Change in Eczema Area and Severity Index (EASI75) [Time Frame: Baseline and 48 weeks]
Proportion of patients with EASI75 (> of equal to 75% reduction from baseline EASI score) at 48 weeks compared to baseline. The EASI was used to assess the severity and extent of AD. Four AD disease characteristics were assessed (0=absent to 3=severe). Area of AD involvement was assessed as a percentage by involved body area of the head, trunk, arms, and legs, and converted to a score of 0 to 6.
Change in Eczema Area and Severity Index (EASI50) [Time Frame: Baseline and 48 weeks]
Proportion of patients with EASI50 (> of equal to 50% reduction from baseline EASI score) at 48 weeks compared to baseline. The EASI was used to assess the severity and extent of AD. Four AD disease characteristics were assessed (0=absent to 3=severe). Area of AD involvement was assessed as a percentage by involved body area of the head, trunk, arms, and legs, and converted to a score of 0 to 6.
Number of Adverse events [Time Frame: 72 weeks]
Safety profile of dupilumab in subjects with AA by reported by number adverse effects.

Study Results. Of 65 screened patients, 60 were randomized in a 2:1 ratio to receive dupilumab (n=40) or placebo (n=20). At week 24, 34 (85%) and 17 (85%) patients on dupilumab and placebo, respectively, completed treatment. In the open-label phase of this trial, from week 24 to week 48, 32 (94%) and 12 (70.6%) patients on dupilumab and placebo, respectively, completed treatment. The mean age was 44 years, 71.7% were women, 76.7% were white, the mean duration since last hair regrowth was 3.7 years, and 36.7% of patients had AT/AU (Table 1). Twenty-three patients (38.3%) had a history of AD, out of which seven (11.7%) had active AD at baseline, 27 (45%) participants had a family history of an atopic disease (eg, AD, asthma), and 18 (30%) had high total serum IgE levels of ≥200 IU/ml. There were no significant differences in baseline patient characteristics between drug and placebo arms (Table 1).

TABLE 1
Patient Baseline Characteristics

| | Placebo | Dupilumab |
|---|---|---|
| | (N = 20) | (N = 40) |
| Mean age, years (SD) | 46.5 (14.4) | 41.6 (13.8) |
| Female sex, N (%) | 13 (65%) | 30 (75%) |
| Race, N (%) | | |
| White | 15 (75%) | 31 (77.5%) |
| African American | 2 (10%) | 3 (7.5%) |
| Asian | 2 (10%) | 6 (15%) |
| Other | 1 (5%) | 0 (0%) |
| Mean duration since last hair | 3.5 (3) | 3.8 (2.9) |
| regrowth, years (SD) | | |
| Mean SALT (SD) | 75.4 (26.1) | 70.5 (27.6) |
| Patients with SALT > 75, N (%) | 12 (60%) | 20 (50%) |
| Patients with SALT < 75, N (%) | 8 (40%) | 20 (50%) |
| Patients with Alopecia Totalis/ | 8 (40%) | 13 (32.5%) |
| Universalis, N (%) | | |
| Mean AASSIS score (SD) | 56.1 (33.8) | 48.4 (30.7) |
| Mean AA-QoL score (SD) | 51.8 (14.2) | 49.5 (12.7) |
| Mean eyelash assessment, N (%) | | |
| Very prominent | 6 (30) | 12 (30) |
| Prominent | 2 (10) | 6 (15) |
| Moderate | 1 (5) | 4 (10) |
| Minimal | 6 (30) | 9 (22.5) |
| None | 4 (20) | 9 (22.5) |
| Mean eyebrow assessment, N (%) | | |
| Very prominent | 6 (30) | 10 (25) |
| Prominent | 0 (0) | 5 (12.5) |
| Moderate | 2 (10) | 6 (15) |
| Minimal | 8 (40) | 9 (22.5) |
| None | 4 (20) | 10 (25) |
| Patients with AD history, N (%) | 6 (30%) | 17 (42.5%) |
| Patients with active AD, N (%) | 2 (10%) | 5 (12.5%) |
| Mean EASI score (SD) | 27.4 (15.6) | 13.6 (6.4) |
| Patients with family history of atopy, | 9 (45%) | 18 (45%) |
| N (%) | | |
| Mean IgE, IU/ml (SD) | 342.5 (826.7) | 525.8 (1211.3) |
| Patients with IgE ≥ 200 IU/ml, | 5 (25%) | 13 (32.5%) |
| N (%) | | |

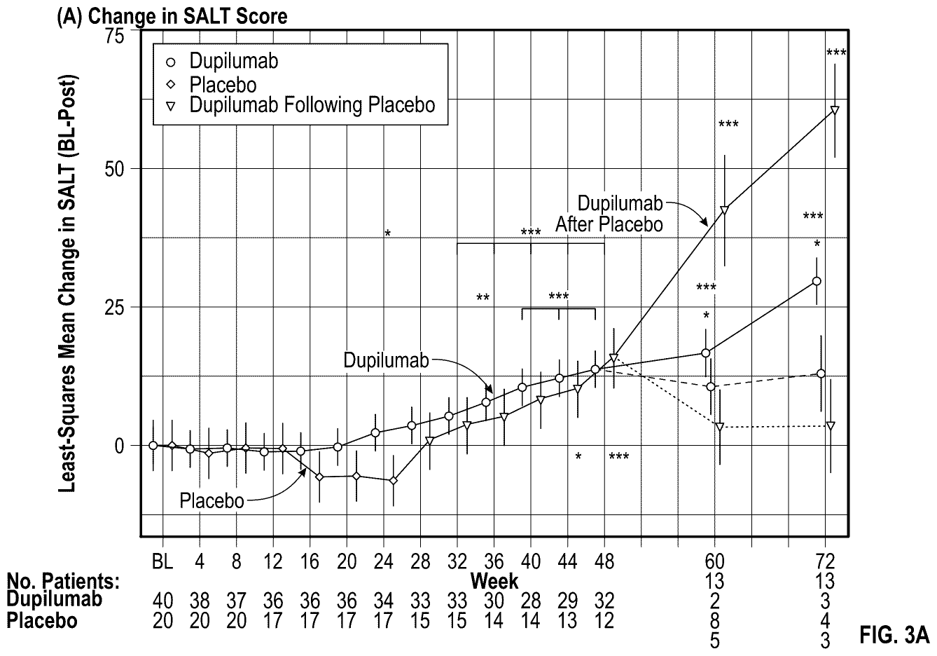

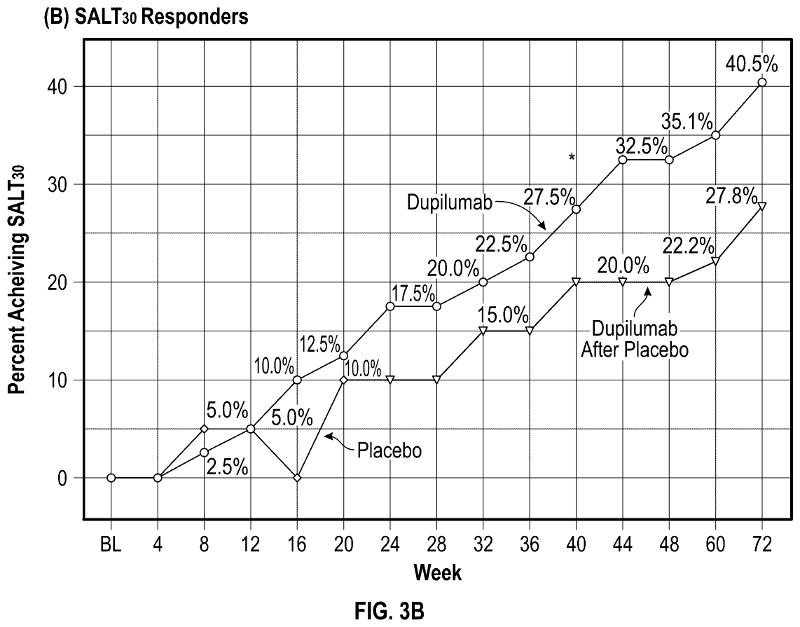

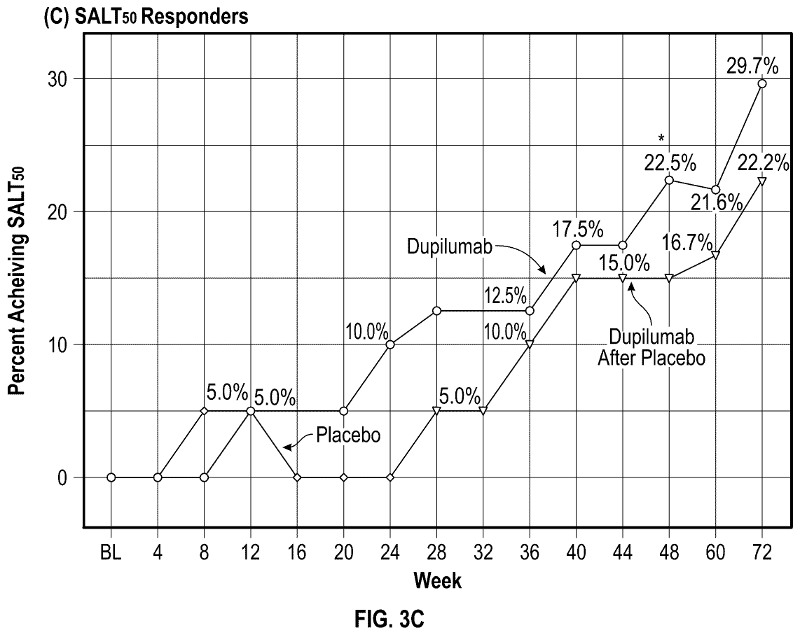

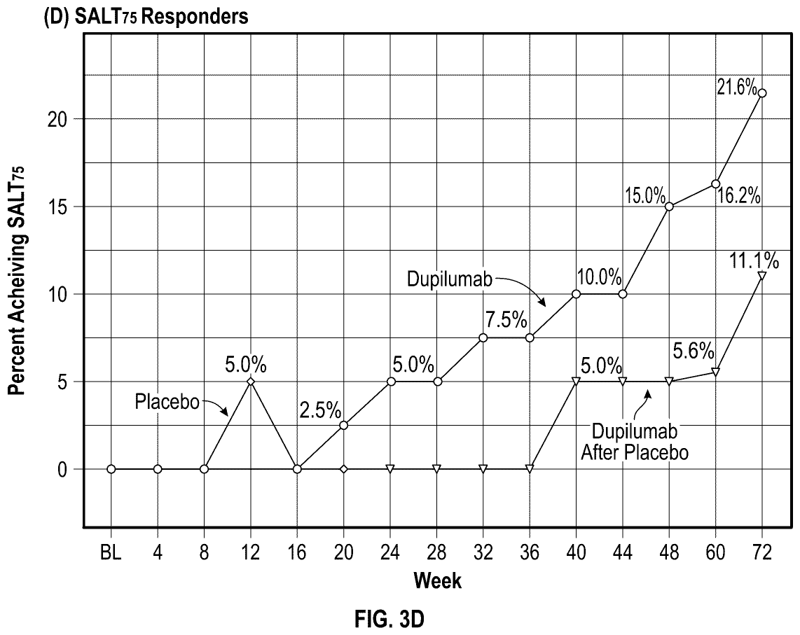

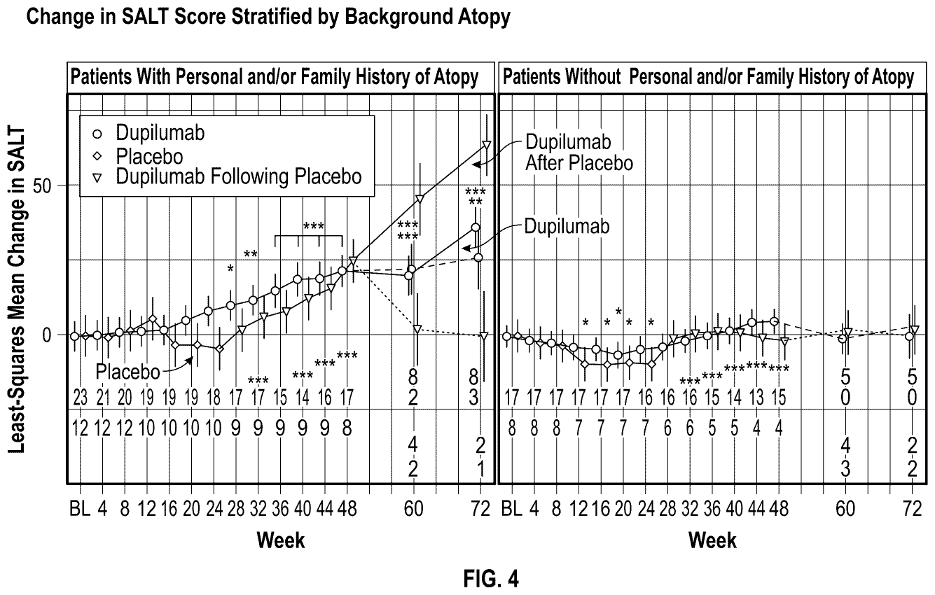

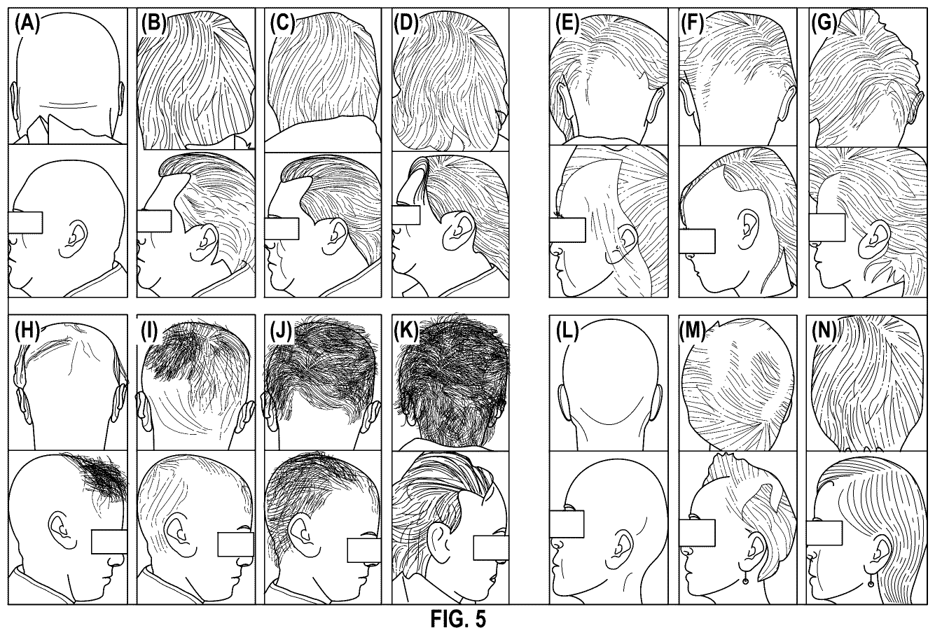

At week 24, worsening of AA was documented in the placebo group, with a least-squares mean change in the SALT score of −6.3 (95% Cl, −14.6 to 1.8), while in the drug arm we observed a least-squares mean change of 2.3 (95% Cl, −3.5 to 8.2) as compared to baseline (p<0.05). Change in least-squares mean SALT score from baseline to week 48 in the drug arm was 13.7 (95% Cl, 7.8 to 19.7) compared to baseline (p<0.001). Change in least-squares mean SALT score from week 24 to week 48 in the placebo arm was 15.9 (95% CI 6.7 to 25.1) compared to placebo arm at week 24, when the open-label phase started (p<0.001). Change in least-squares mean SALT score from week 24 to week 48 in the drug arm versus the change in least-squares mean SALT score in the placebo arm from baseline to week 24 was 17.4 (95% Cl: 2.84-31.93) (p=0.019). Change in least-squares mean SALT score from week 24 to week 48 in the placebo arm versus the change in least-squares mean SALT score in the placebo arm from baseline to week 24 was 22.3 (95% Cl: 12.1-32.5) (p<0.001) (Table 2 and FIG. 3A). When patients were stratified based on personal and/or family history of atopy, baseline SALT scores were not different between atopic and non-atopic participants. However, significant improvement in least-squares mean SALT score was detected much earlier in atopic as compared to non-atopic patients, that achieved significance at week 48 (FIG. 4). Clinical pictures of participants are shown in FIG. 5.

TABLE 2
Efficacy Outcomes

| | Placebo | Dupilumab | |
|---|---|---|---|
| | (N = 17 at | (N = 34 at week | |
| | week 24; N = 12 at | 24; N = 32 at | P - |
| | week 48) | week 48) | value |
| Change in SALT from baseline | −6.3 (4.2) | 2.3 (3) | .049 |
| to week 24 (SE)* | | | |
| Key secondary endpoints: | | | |
| Change in SALT from baseline | NA | 13.7 (3) | <.0001 |
| to week 48 in the drug-first | | | |
| arm (SE)* | | | |
| Change in SALT from week 24 | 15.9 (4.7) | NA | .0006 |
| to week 48 in the placebo-first | | | |
| arm (SE)* | | | |
| Change in SALT from week 24 | 22.3 (5.2) | NA | <.0001 |
| to week 48 in the placebo-first | | | |
| arm versus change in SALT | | | |
| from baseline to week 24 in | | | |
| the placebo-first arm (SE)* | | | |
| Change in SALT from week 24 | NA | 17.4 (7.3) | .019 |
| to week 48 in the placebo-first | | | |
| arm versus change in SALT | | | |
| from baseline to week 24 in | | | |
| the drug-first arm (SE)* | | | |

Proportion of patients achieving SALT_{30/50/75/90} score at
week 24, N (%)

| | | | |
|---|---|---|---|
| ≥30% (SALT₃₀) | 2 (10%) | 7 (17.5%) | .66 |
| ≥50% (SALT₅₀) | 0 (0%) | 4 (10%) | .23 |
| ≥75% (SALT₇₅) | 0 (0%) | 2 (5%) | .55 |
| ≥90% (SALT₉₀) | 0 (0%) | 1 (2%) | 1 |

Proportion of patients achieving SALT_{30/50/75/90} in drug-first arm
at week 48 compared to placebo-first arm at week 24, N (%)

| | | | |
|---|---|---|---|
| ≥30% (SALT₃₀) | 4 (20%) | 13 (32.5%) | .067 |
| ≥50% (SALT₅₀)* | 3 (15%) | 9 (22.5%) | .02 |
| ≥75% (SALT₇₅) | 1 (5%) | 6 (15%) | .17 |
| ≥90% (SALT₉₀) | 1 (5%) | 4 (10%) | .29 |

Proportion of patients achieving SALT_{30/50/75/90} in
placebo-first arm at week 48 compared to
placebo-first arm at week 24, N (%)

| | Week | Week | |
|---|---|---|---|
| | 24 | 48 | |
| ≥30% (SALT₃₀) | 2 (10) | 4 (20) | NA |
| ≥50% (SALT₅₀) | 0 (0) | 3 (15) | NA |
| ≥75% (SALT₇₅) | 0 (0) | 1 (5) | NA |
| ≥90% (SALT₉₀) | 0 (0) | 1 (5) | NA |

Proportion of drug-first arm patients achieving
SALT_{30/50/75/90} at week 48 compared to placebo-
first arm patients at week 24, N (%)

| | | |
|---|---|---|
| ≥30% (SALT₃₀) | 2 (10) | 17 (28.3) |
| ≥50% (SALT₅₀)* | 0 (0) | 12 (20) |
| ≥75% (SALT₇₅) | 0 (0) | 7 (12) |
| ≥90% (SALT₉₀) | 0 (0) | 5 (8) |

Upon grouping all patients treated with 24 weeks of dupilumab together (i.e., those completing week 24 in the drug arm with those completing weeks 24 to 48 in the placebo arm, after switching to dupilumab), SALT₃₀ was achieved by 18.3% of patients, compared with 10% in the placebo group. The proportions of patients achieving SALT₅₀ and SALT₇₅ were 11.7% and 5%, respectively, versus 0% (for both SALT₅₀ and SALT₇₅) in the placebo group (FIGS. 3B-3D) (p<0.05 for SALT₅₀). By week 48, 32.5% of the patients achieved SALT₃₀, 22.5% achieved SALT₅₀, and 15% achieved SALT₇₅ in the drug arm (Table 2).

Baseline characteristics of patients achieving SALT₃₀ were compared with patients that did not achieve SALT₃₀ after 24 weeks of dupilumab treatment in both study arms (first 24 weeks for drug-first arm and weeks 24-48 in the placebo-first arm). Baseline SALT scores, IgE levels, and the proportion of patients with concomitant AD/AD history or with a family history of atopy were significantly different among SALT₃₀ responders and non-responders (Table 3 and Table 4) (p<0.05).

TABLE 3
Baseline Characteristics of Patients Treated with Dupilumab
for 24 Weeks by Response (Both Study Arms)

| | | | | | | | | | | All patients | |
|---|---|---|---|---|---|---|---|---|---|---|---|
| | | | | | | | | | | completing | |
| | Non- | | Responders | | | Responders | | Responders | | first 24 | |
| | Responders | | SALT₃₀ | | | SALT₅₀ | | SALT₇₅ | | weeks of | |
| | (SALT₀₋₃₀), | | and up, | | P- | and up, | | and up, | | drug (ITT), | |
| | N = 49 | | N = 11 | | value | N = 7 | | N = 3 | | N = 60 | |
| Mean age, | 43 | (14) | 46 | (17) | 0.70 | 41 | (18) | 39 | (17) | 43 | (13) |
| years (SD) | | | | | | | | | | | |
| Female | 33 | (67.3%) | 10 | (90.9%) | 0.24 | 6 | (85.7%) | 2 | (66.7%) | 43 | (71.7%) |
| sex, N (%) | | | | | | | | | | | |
| Race, N | | | | | | | | | | | |
| (%) | | | | | | | | | | | |
| White | 40 | (81.6) | 7 | (63.6) | 0.42 | 5 | (71.4) | 2 | (66.7) | 46 | (76.7) |
| African | 3 | (6.1) | 2 | (18.2) | 0.24 | 0 | (0) | 0 | (0) | 5 | (8.3) |
| American | | | | | | | | | | | |
| Asian | 6 | (12.2) | 2 | (18.2) | 0.99 | 2 | (28.6) | 1 | (33.3) | 8 | (13.3) |
| Mean | 3.8 | (3.1) | 2.6 | (2.1) | 0.086 | 2.4 | (2.4) | 1.3 | (1.9) | 3.6 | (3) |
| duration | | | | | | | | | | | |
| since last | | | | | | | | | | | |
| hair | | | | | | | | | | | |
| regrowth, | | | | | | | | | | | |
| years (SD) | | | | | | | | | | | |
| Mean | 76.3 | (26) | 57.9 | (24.1) | 0.03 | 60.4 | (28.1) | 63.6 | (31.2) | 72.1 | (27) |
| baseline | | | | | | | | | | | |
| SALT | | | | | | | | | | | |
| (SD)* | | | | | | | | | | | |
| Patients | 20 | (40.8%) | 8 | (72.7%) | 0.17 | 4 | (57.1%) | 2 | (66.7%) | 28 | (46.7%) |
| with | | | | | | | | | | | |
| baseline | | | | | | | | | | | |
| SALT < 75, | | | | | | | | | | | |
| N | | | | | | | | | | | |
| Patients | 29 | (59.2%) | 3 | (27.3%) | | 3 | (42.9%) | 1 | (33.3%) | 32 | (53.3%) |
| with | | | | | | | | | | | |
| baseline | | | | | | | | | | | |
| SALT > 75, | | | | | | | | | | | |
| N | | | | | | | | | | | |
| Patients | 23 | (46.9%) | 6 | (54.5%) | 0.30 | 5 | (71.4%) | 2 | (66.7%) | 29 | (48.3%) |
| with AD | | | | | | | | | | | |
| history, N | | | | | | | | | | | |
| Patients | 19 | (38.8%) | 8 | (72.7%) | 0.08 | 6 | (85.7%) | 2 | (66.7%) | 27 | (45%) |
| with | | | | | | | | | | | |
| family | | | | | | | | | | | |
| history of | | | | | | | | | | | |
| atopy, N | | | | | | | | | | | |
| (%) | | | | | | | | | | | |
| Patients | 15 | (30.6%) | 10 | (90.9%) | 0.01 | 7 | (100%) | 3 | (100%) | 35 | (58.3%) |
| with | | | | | | | | | | | |
| personal | | | | | | | | | | | |
| or family | | | | | | | | | | | |
| history of | | | | | | | | | | | |
| atopy, N | | | | | | | | | | | |
| Patients | 12 | (24.5%) | 7 | (63.6%) | 0.01 | 6 | (85.7%) | 3 | (100%) | 19 | (30%) |
| with | | | | | | | | | | | |
| IgE > 200 | | | | | | | | | | | |
| IU/ml at | | | | | | | | | | | |
| baseline, | | | | | | | | | | | |
| N | | | | | | | | | | | |
| Mean | 338.8 | (976.4) | 946.2 | (1450) | 0.04 | 6 | (85.7) | 487.7 | (301.3) | 464.7 | (1094) |
| baseline | | | | | | | | | | | |
| IgE, IU/ml | | | | | | | | | | | |
| (SD)* | | | | | | | | | | | |
| History of | 44 | (89.8%) | 10 | (90.9%) | 1 | 6 | (85.7%) | 3 | (100%) | 54 | (90%) |
| previous | | | | | | | | | | | |
| AA | | | | | | | | | | | |
| treatments, | | | | | | | | | | | |
| N (%) | | | | | | | | | | | |
| History of | 17 | (34.7%) | 3 | (27.2%) | 0.32 | 2 | (28.6%) | 1 | (33.3%) | 20 | (33.3%) |
| previous | | | | | | | | | | | |
| systemic | | | | | | | | | | | |
| AA | | | | | | | | | | | |
| treatments, N | | | | | | | | | | | |
| Mean | 2.9% | (1.9) | 4.8% | (4.1) | 0.15 | 5.6% | (4.6) | 5.4% | (3.6) | 3.2% | (2.5) |
| baseline | | | | | | | | | | | |
| eosinophils, (SD) | | | | | | | | | | | |
| Mean | 191.1 | (36.7) | 211 | (59.4) | 0.61 | 233.7 | (48.1) | 216 | (46.1) | 194.5 | (43.2) |
| baseline | | | | | | | | | | | |
| LDH (SD) | | | | | | | | | | | |
| Mean | 1.6 | (2.8) | 1.5 | (1.7) | 1 | 2.2 | (1.9) | 1.2 | (1) | 1.8 | (2.9) |
| baseline | | | | | | | | | | | |
| CRP (SD) | | | | | | | | | | | |

TABLE 4
Baseline Characteristics of Patients by Response after 48 Weeks of Trial

| | | | | | | | | | | | | Per- | |
|---|---|---|---|---|---|---|---|---|---|---|---|---|---|
| | Non- | | | | | | | | | | | protocol | |
| | Responders | | | | | | | | | | | cohort | |
| | at week 48 | | Responders | | | Responders | | Responders | | Responders | | (completing | |
| | (SALT₀₋₃₀), | | SALT₃₀, | | P- | SALT₅₀, | | SALT₇₅, | | SALT₉₀, | | week 48), | |
| | N = 27 | | N = 17 | | value | N = 12 | | N = 7 | | N = 4 | | N = 44 | |
| Mean | 43 | (13) | 43 | (16) | 0.78 | 44 | (18) | 42 | (12) | 42 | (19) | 43 | (13) |
| age, | | | | | | | | | | | | | |
| years | | | | | | | | | | | | | |
| (SD) | | | | | | | | | | | | | |
| Female | 29 | (67.4%) | 11 | (64.7%) | 1 | 10 | (83.3%) | 6 | (85.7%) | 3 | (75%) | 43 | (71.7%) |
| sex, N | | | | | | | | | | | | | |
| (%) | | | | | | | | | | | | | |
| Race, N | | | | | | | | | | | | | |
| White | 43 | (13%) | 11 | (64.7%) | 0.30 | 8 | (66.7%) | 4 | (57.1%) | 3 | (75%) | 46 | (76.7%) |
| African | 29 | (67.4%) | 2 | (11.8%) | 0.60 | 1 | (8.3%) | 0 | (0%) | 0 | (0%) | 5 | (8.3%) |
| American | | | | | | | | | | | | | |
| Asian | 43 | (13%) | 4 | (23.5%) | 0.40 | 3 | (25%) | 3 | (42.9%) | 1 | (25%) | 8 | (13.3%) |
| Mean | 29 | (67.4) | 3.1 | (2.6) | 0.48 | 2.5 | (1.9) | 2.6 | (2.1) | 1.13 | (1%) | 3.6 | (3) |
| duration | | | | | | | | | | | | | |
| since | | | | | | | | | | | | | |
| last hair | | | | | | | | | | | | | |
| regrowth, | | | | | | | | | | | | | |
| years | | | | | | | | | | | | | |
| (SD) | | | | | | | | | | | | | |
| Mean | 43 | (13) | 56.3 | (23.4) | 0.0007 | 57.6 | (25.5) | 53.4 | (29.5) | 46.9 | (32.1) | 72.1 | (27) |
| baseline | | | | | | | | | | | | | |
| SALT | | | | | | | | | | | | | |
| (SD) | | | | | | | | | | | | | |
| Patients | 29 | (67.4%) | 12 | (70.6%) | 0.03 | 8 | (66.7%) | 5 | (71.4%) | 3 | (75%) | 28 | (46.7%) |
| with | | | | | | | | | | | | | |
| baseline | | | | | | | | | | | | | |
| SALT < 75, N | | | | | | | | | | | | | |
| Patients | 43 | (13%) | 5 | (39.4%) | | 4 | (33.3%) | 2 | (28.6%) | 1 | (25%) | 32 | (53.3%) |
| with | | | | | | | | | | | | | |
| baseline | | | | | | | | | | | | | |
| SALT > 75, N | | | | | | | | | | | | | |
| Patients | 29 | (67.4%) | 8 | (47%) | 0.53 | 7 | (58.3%) | 5 | (71.4%) | 3 | (75%) | 29 | (48.3%) |
| with AD | | | | | | | | | | | | | |
| history, | | | | | | | | | | | | | |
| N | | | | | | | | | | | | | |
| Patients | 43 | (13%) | 10 | (58.8%) | 0.22 | 7 | (58.3) | 5 | (71.4) | 2 | (50) | 27 | (45) |
| with | | | | | | | | | | | | | |
| family | | | | | | | | | | | | | |
| history | | | | | | | | | | | | | |
| of atopy, N | | | | | | | | | | | | | |
| Patients | 29 | (67.4%) | 12 | (70.1%) | 0.21 | 9 | (75%) | 6 | (85.7%) | 3 | (75%) | 35 | (58.3%) |
| with | | | | | | | | | | | | | |
| personal | | | | | | | | | | | | | |
| or | | | | | | | | | | | | | |
| family | | | | | | | | | | | | | |
| history | | | | | | | | | | | | | |
| of atopy, N | | | | | | | | | | | | | |
| Patients | 43 | (13%) | 9 | (52.9%) | 0.02 | 8 | (66.7%) | 6 | (85.7%) | 3 | (75%) | 19 | (30%) |
| with | | | | | | | | | | | | | |
| IgE > 200 | | | | | | | | | | | | | |
| IU/ml at | | | | | | | | | | | | | |
| baseline, N | | | | | | | | | | | | | |
| Mean | 29 | (67.4) | 660.8 | (1215) | 0.01 | 844.3 | (1404) | 863.7 | (1408) | 400 | (352.2) | 464.7 | (1094) |
| baseline | | | | | | | | | | | | | |
| IgE, | | | | | | | | | | | | | |
| IU/ml | | | | | | | | | | | | | |
| (SD) | | | | | | | | | | | | | |
| History | 43 | (13%) | 17 | (100%) | 0.15 | 12 | (100%) | 7 | (100%) | 4 | (100%) | 54 | (90%) |
| of | | | | | | | | | | | | | |
| previous | | | | | | | | | | | | | |
| AA | | | | | | | | | | | | | |
| treatments, N | | | | | | | | | | | | | |
| History | 29 | (67.4) | 5 | (29.4) | 0.75 | 4 | (33.3) | 3 | (42.9) | 2 | (50) | 20 | (33.3) |
| of | | | | | | | | | | | | | |
| previous | | | | | | | | | | | | | |
| systemic | | | | | | | | | | | | | |
| AA | | | | | | | | | | | | | |
| treatments, N | | | | | | | | | | | | | |
| Mean | 43 | (13) | 4.24 | (3.46) | 0.16 | 4.3 | (3.9) | 5.7 | (4.6) | 2.9 | (2.3) | 3.2 | (2.5) |
| baseline | | | | | | | | | | | | | |
| eosinophils % | | | | | | | | | | | | | |
| (SD) | | | | | | | | | | | | | |
| Mean | 29 | (67.4) | 196.9 | (51.8) | 0.94 | 208.2 | (56.8) | 250.3 | (64.2) | 162 | (36.5) | 194.5 | (43.2) |
| baseline | | | | | | | | | | | | | |
| LDH | | | | | | | | | | | | | |
| (SD) | | | | | | | | | | | | | |
| Mean | 43 | (13) | 1.2 | (1.6) | 0.23 | 1.6 | (1.7) | 1.2 | (1.3) | 0.6 | (0.7) | 1.8 | (2.9) |
| baseline | | | | | | | | | | | | | |
| CRP | | | | | | | | | | | | | |
| (SD) | | | | | | | | | | | | | |

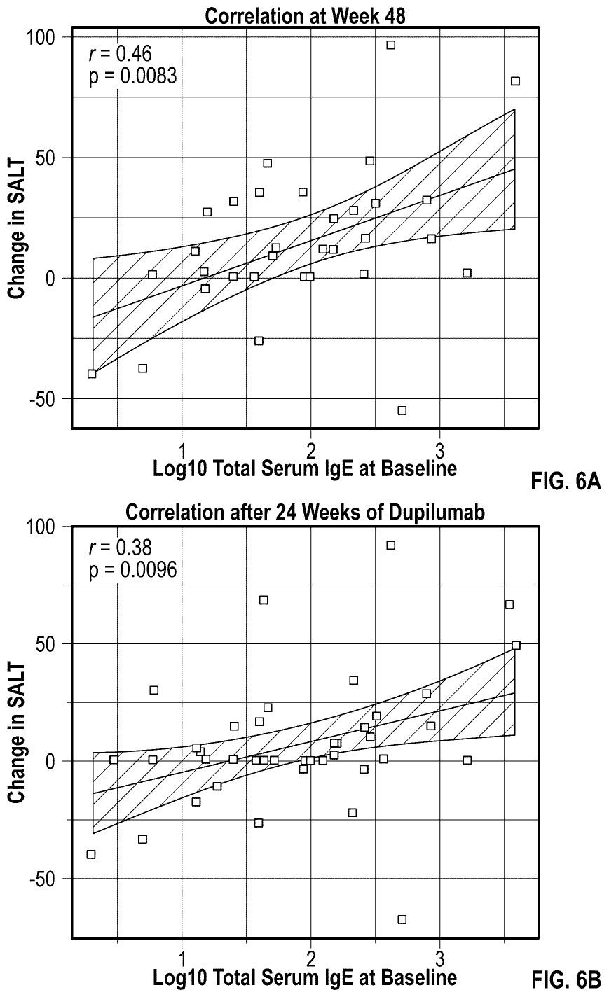

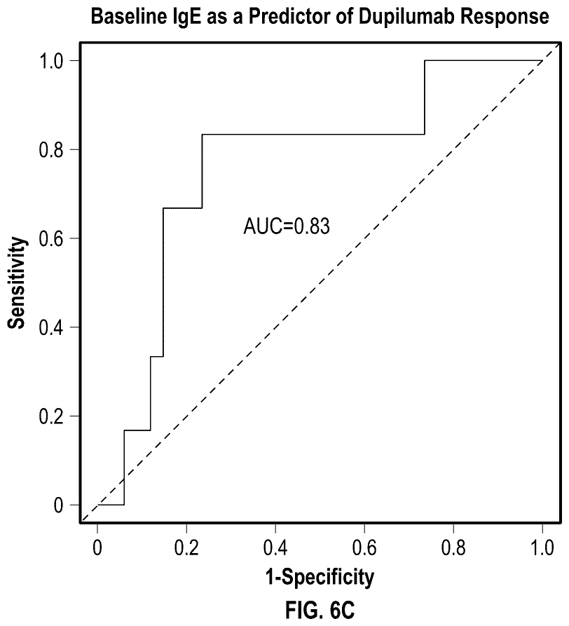

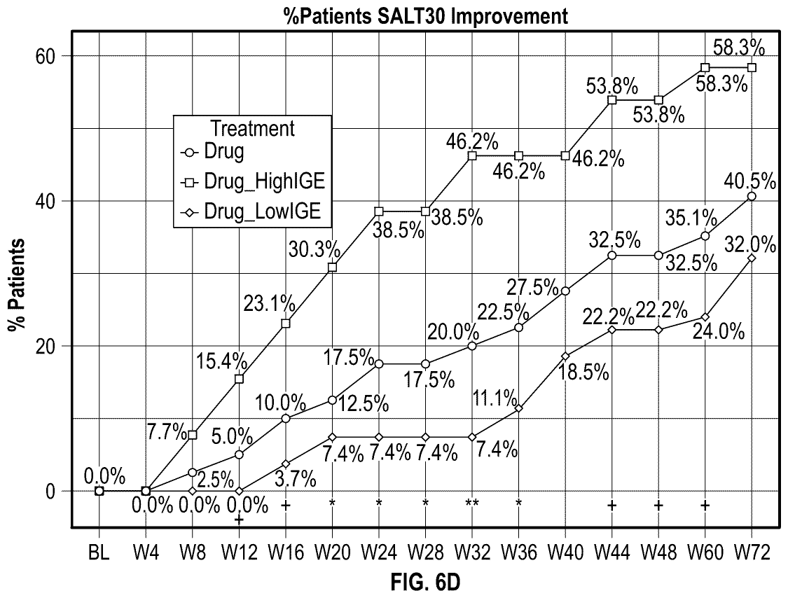

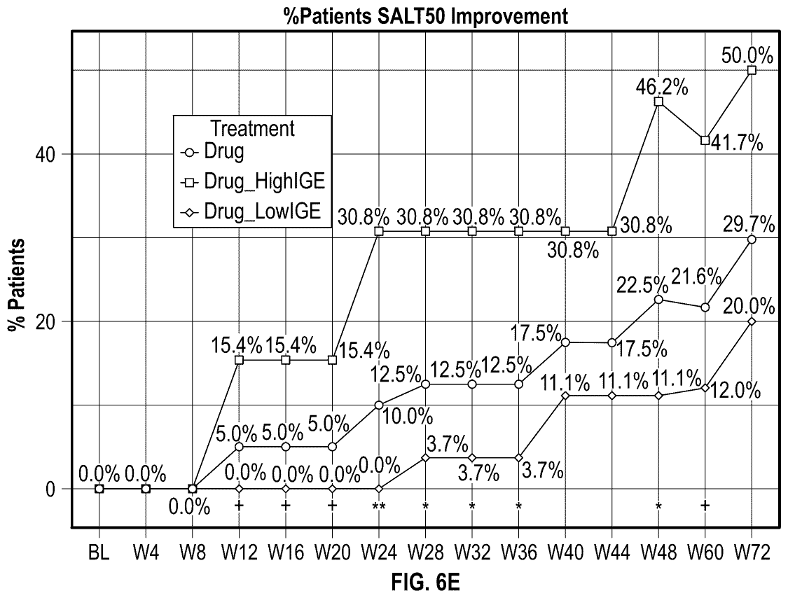

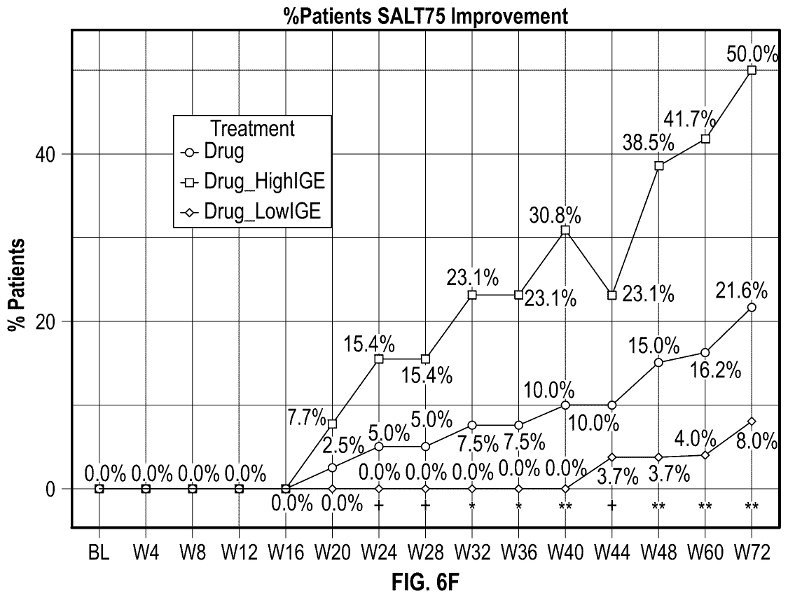

Patients achieving SALT₃₀ had significantly higher baseline IgE levels (946.2, 95% Cl: 0-2,460.2) versus non-responders (338.8, 95% Cl: 0-612.2). Moreover, change in SALT and IgE levels showed a robust and significant correlation both after 48 weeks in the drug arm (r=0.46, 95% Cl: 0.13-0.7, p=0.0083), as well as after 24 weeks of dupilumab treatment in both study arms (r=0.38, 95% Cl: 0.099-0.6, p=0.0096). Further, baseline serum IgE predicts dupilumab response with an AUC of 0.83 (FIGS. 6A-6F). To further clarify the possible utility of IgE to predict dupilumab response and potentially improve patient selection, we evaluated response in the entire dupilumab arm as compared to those patients with high and low baseline IgE (IgE 200 IU/ml) in this arm (FIGS. 6A-6F, Table 5, and Table 6).

TABLE 5
Proportion of patients achieving SALT
at week 24 based on baseline IgE level
Proportion of patients achieving SALT_{30/50/75/90}
in the drug arm at week 24 based on
baseline IgE level (threshold: 200 IU/ml)

| | Low IgE | High IgE | Entire drug arm | |
|---|---|---|---|---|
| | (N = 27) | (N = 13) | (N = 40) | |
| ≥30% (SALT₃₀) | 2 (7.4%) | 5 (38.5%) | 7 (17.5%) | .03 |
| ≥50% (SALT₅₀) | 0 (0%) | 4 (30.8%) | 4 (10%) | .008 |
| ≥75% (SALT₇₅) | 0 (0%) | 2 (15.4%) | 2 (5%) | .1 |
| ≥90% (SALT₉₀)^{c} | 0 (0%) | 1 (7.7%) | 1 (2%) | .32 |

TABLE 6
Proportion of patients achieving SALT
at week 48 based on baseline IgE level
Proportion of patients achieving SALT_{30/50/75/90}
in the drug arm at week 48 based on
baseline IgE level (threshold: 200 IU/ml)^{b}

| | Low IgE | High IgE | All drug arm | |
|---|---|---|---|---|
| | (N = 27) | (N = 13) | (N = 40) | |
| ≥30% (SALT₃₀) | 6 (22.2%) | 7 (53.8%) | 13 (32.5%) | .07 |
| ≥50% (SALT₅₀) | 3 (11.1%) | 6 (46.2%) | 9 (22.5%) | .04 |
| ≥75% (SALT₇₅) | 1 (3.7%) | 5 (38.5%) | 6 (15%) | .009 |
| ≥90% (SALT₉₀)^{c} | 1 (3.7%) | 3 (23.1%) | 4 (10%) | .09 |

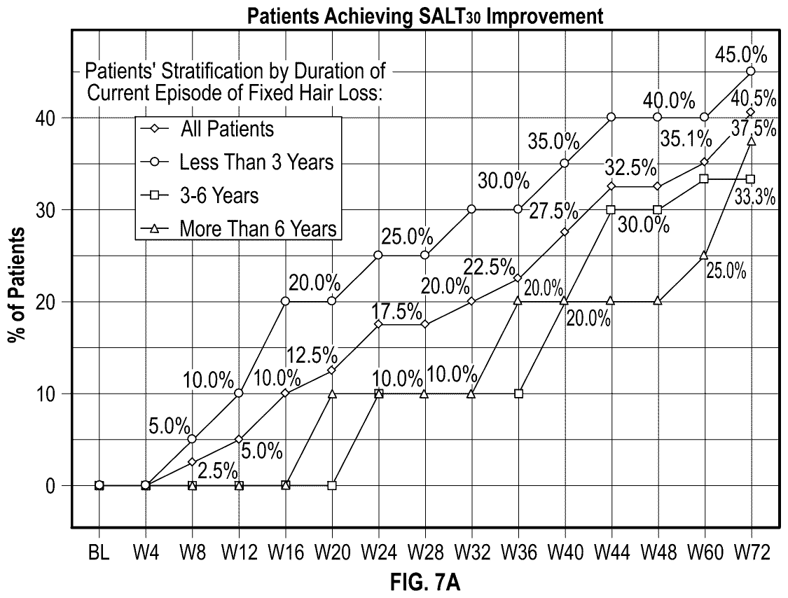

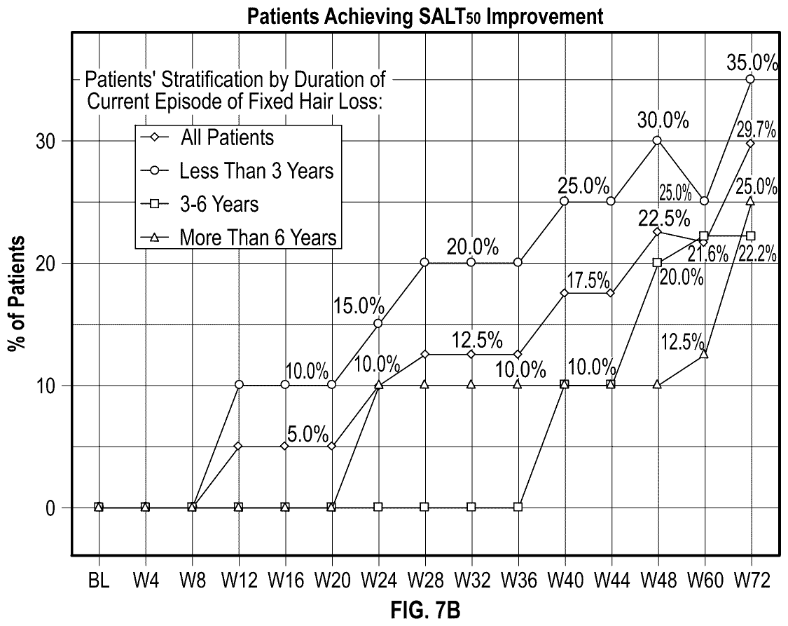

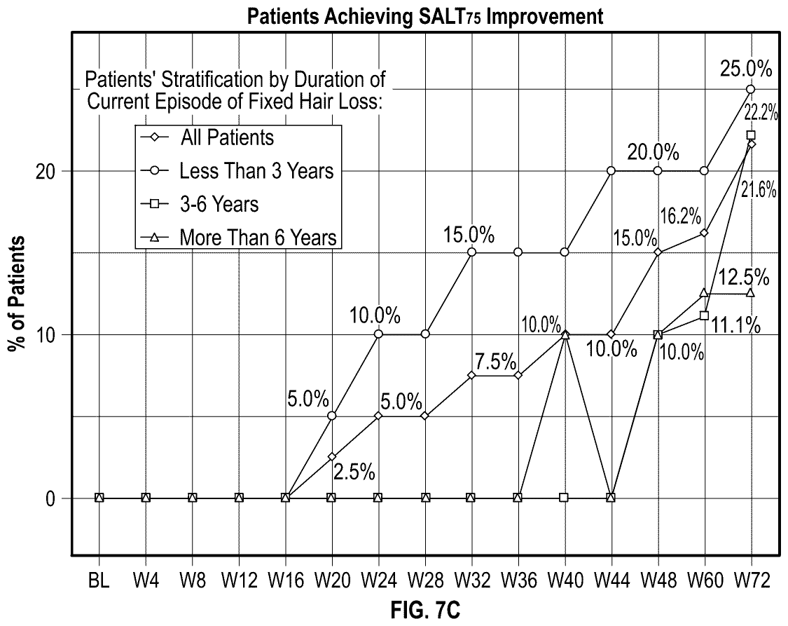

Patients with high IgE had the highest SALT_{30/50/75/90} response rates compared to the other groups (FIGS. 3A-3F). In fact, patients with high IgE had an odd ratio of 8.1 to achieve SALT₃₀ response as compared with patients with low IgE at week 24. Concomitant AD/AD history or a family history of atopy were found in 90.9% of patients achieving SALT₃₀ but only in 30.6% of non-responders (p<0.05). Greater response to dupilumab treatment was observed in patients with shorter periods since last hair regrowth, as shown in FIGS. 7A-7C. Other baseline characteristics, including age, gender, race, mean duration since last hair regrowth, or other blood markers (i.e., CRP, LDH, eosinophil percentage) were not significantly different among SALT₃₀ responders and non-responders.

Of patients with eyelash or eyebrow hair loss at baseline (a grade of in a 0-4 scale), 18.2% and 27.3% achieved grade improvement in the eyelash assessment and 7.1% and 20% achieved grade improvement in the eyebrow assessment at week 24 compared to baseline in the placebo and drug arms, respectively. These proportions increased to 27.3% and 31.8% for eyelash assessment and 28.6% and 24% for eyebrow assessment at week 48 compared to baseline in the placebo and drug arms, respectively (Table 7).

TABLE 7
Additional Efficacy Outcomes

| | Placebo | Dupilumab | |
|---|---|---|---|
| | (N = 17 at Week | (N = 34 at Week | |
| | 24; N = 12 at | 24; N = 32 at | |
| | Week 48 for AA. | Week 48 for AA. | P- |
| | N = 2 for AD) | N = 3 for AD) | value |
| Change in AASIS score | 10.25 (20.29) | 0.64 (22.27) | NA |
| at week 24 compared to | | | |
| baseline (SE) | | | |
| Change in AASIS score | 22.6 (35.42) | 8.13 (27.65) | NA |
| at week 48 compared to | | | |
| baseline (SE) | | | |
| Change in AASIS score | 10.25(20.29) | 8.13 (27.65) | NA |
| at week 48 compared to | | | |
| baseline in drug arm | | | |
| versus change in AASIS | | | |
| score at week 24 | | | |
| compared to baseline in | | | |
| placebo arm (SE) | | | |
| Change in AA-QoL score | 4.29 (9.14) | 2.06 (8.49) | NA |
| at week 24 compared to | | | |
| baseline (SE) | | | |
| Change in AA-QoL score | 22.6 (35.42) | 8.13 (27.65) | NA |
| at week 48 compared to | | | |
| baseline (SE) | | | |
| Change in AA-QoL score | 4.29 (9.14) | 8.13 (27.65) | NA |
| at week 48 compared to | | | |
| baseline in Drug arm | | | |
| versus change in AA- | | | |
| QoL score at week 24 | | | |
| compared to baseline in | | | |
| Placebo arm (SE) | | | |
| Change in EASI score at | −1.45 (3.61) | 10.6 (3.64) | 0.05 |
| week 24 compared to | | | |
| baseline (SE) | | | |
| Change in EASI score at | 23.9 (10.61) | 10.53 (3.75) | 0.31 |
| week 48 compared to | | | |
| baseline (SE) | | | |
| Change in EASI score at | −1.45 (3.61) | 10.53 (3.75) | 0.05 |
| week 48 compared to | | | |
| baseline in drug arm | | | |
| versus change in EASI | | | |
| score at week 24 | | | |
| compared to baseline in | | | |
| placebo arm (SE) | | | |
| Proportion of patients | 11.76% | 20.59% | 0.70 |
| with AA-PGA score >1 at | | | |
| week 24, % | | | |
| Proportion of patients | 25% | 46.87% | 0.30 |
| with AA-PGA score >1 at | | | |
| week 48, % | | | |
| Proportion of patients | 18.18 | 27.27 | 0.69 |
| with eyelash score >1 | | | |
| improvement at week 24 | | | |
| Proportion of patients | 27.27 | 31.81 | 0.68* |
| with eyelash score >1 | | | |
| improvement at week 48 | | | |
| Proportion of patients | 7.1 | 20 | 0.39 |
| with eyebrow score >1 | | | |
| improvement at week 24 | | | |
| Proportion of patients | 28.6 | 24 | 0.38* |
| with eyebrow score >1 | | | |
| improvement at week 48 | | | |

Baseline characteristics of patients achieving SALT₃₀ were compared with patients that did not achieve SALT₃₀ after 24 weeks of dupilumab treatment in both study arms (first 24 weeks for drug-first arm and weeks 24-48 in the placebo-first arm). Baseline SALT scores, IgE levels, and the proportion of patients with concomitant AD/AD history or with a family history of atopy were significantly different among SALT₃₀-responders or non-responders (Table 3). Patients achieving SALT₃₀ had significantly higher baseline IgE levels (946.2, 95% Cl, 0 to 2,460.2) versus non-responders (145.9, 95% Cl, 0 to 440.1). Moreover, change in SALT and total serum IgE levels showed a robust and significant correlation both after 48 weeks in the drug arm (r=0.46, 95% Cl, 0.13 to 0.7, P=0.0083), as well as after 24 weeks of dupilumab treatment in both study arms (r=0.38, 95% Cl, 0.099 to 0.6, P=0096). Further, baseline serum IgE predicts dupilumab response with an AUC of 0.83. Concomitant AD/AD history or a family history of atopy were found in 90.9% of patients achieving SALT₃₀ but only in 45.7% of non-responders. Other baseline characteristics, including age, gender, race, mean duration since last hair regrowth, or other blood markers (i.e, CRP, LDH, eosinophil percentage) were not significantly different among SALT₃₀ responders and non-responders.

For AA-PGA, another parameter indicating percent of improvement in SALT score compared to baseline, the proportions of patients achieving >1 grade regrowth at week 24 were 5% and 17.5% in the placebo and drug arms, respectively. At week 48, these increased to 20% and 35%, respectively (Table 7).

Although changes in quality-of-life scores (both AA-QLI and AASIS) in drug vs placebo arm did not achieve significance, AASIS scores were significantly lower in responder versus non-responder participants at study endpoints (ie, at both week 24 and week 48), reflecting less impact of AA on participants' quality-of-life. For example, mean AASIS scores reported by responders at week 24 were 15.47 (95% Cl: 1.06-30.08) for those achieving SALT₃₀ and 4.75 (95% Cl: 1.94-7.56) for those achieving SALT₅₀, versus 54.59 (95% Cl: 41.68-67.50) in non-responders, that is, participants with <30% SALT improvement (SALT₀₋₃₀) (p<0.05) (Table 8).

Of the seven patients with active AD, AD severity (by EASI) improved in dupilumab-treated patients after both 24 and 48 weeks of treatment, with no improvement after placebo treatment (Table 8; p<0.05).

TABLE 8
Quality of Life Outcomes by Therapeutic Response

| | | | | | |
|---|---|---|---|---|---|
| | Non- | | Non- | | Non- |
| SALT₃₀ | responders | SALT₅₀ | responders | SALT₇₅ | responders |
| responders | (SALT₀₋₃₀) | responders | (SALT₀₋₅₀) | responders | (SALT₀₋₇₅) |

AASIS at week 24

| | | | | | | |
|---|---|---|---|---|---|---|
| N | 7 | 27 | 4 | 30 | 2 | 32 |
| Mean | 15.57 | 54.59 | 4.75 | 52.13 | 3 | 49.28 |
| (SD) | (19.59) | (34.22) | (2.87) | (33.87) | (0) | (34.63) |

| | | | |
|---|---|---|---|
| P-value | 0.0011 | <0.0001 | <0.0001 |

AA-QLI at week 24

| | | | | | | |
|---|---|---|---|---|---|---|
| N | 7 | 26 | 4 | 29 | 2 | 31 |
| Mean | 40.29 | 47.62 | 36.25 | 47.41 | 41.5 | 46.35 |
| (SD) | (8.69) | (15.92) | (9.88) | (15.07) | (13.44) | (15.13) |

| | | | |
|---|---|---|---|
| P-value | 0.1231 | 0.1044 | 0.6994 |

AASIS at week 48

| | | | | | | |
|---|---|---|---|---|---|---|
| N | 10 | 13 | 7 | 16 | 5 | 18 |
| Mean | 16.3 | 50 | 8.57 | 47.06 | 5.8 | 43.56 |
| (SD) | (18.01) | (29.57) | (8.42) | (28.51) | (4.21) | (28.88) |

| | | | |
|---|---|---|---|
| P-value | 0.003 | 0.0001 | <0.0001 |

AA-QLI at week 48

| | | | | | | |
|---|---|---|---|---|---|---|
| N | 11 | 15 | 8 | 18 | 5 | 21 |
| Mean | 38.64 | 48.4 | 38.88 | 46.67 | 35.8 | 46.29 |
| (SD) | (12.51) | (11.7) | (13.67) | (11.99) | (10.31) | (12.69) |

| | | | |
|---|---|---|---|
| P-value | 0.0564 | 0.1893 | 0.0909 |

AEs were reported in 20% and 25% of patients in the placebo and drug arms, respectively, during the first 24 weeks of this trial, and in 25% and 26.5%, respectively, during weeks 24 to 48 (Table 9).

TABLE 9
Adverse Events

| | | | Placebo/ | |
|---|---|---|---|---|
| | | | Dupilumab | |
| | | | Following | |
| | Drug | | Placebo | |
| | Weeks | Weeks | Weeks | Weeks |
| | 0-24, | 24-48, | 0-24, | 24-48, |
| | N = 40 | N = 34 | N = 20 | N = 17 |
| Total number of adverse | 12 | 12 | 5 | 4 |
| events, N | | | | |
| Patients with adverse events, | 10 (25%) | 9 (26.5%) | 4 (20%) | 3 (25%) |
| N | | | | |
| Patients with serious adverse | 0 | 1 | 0 | 0 |
| events, N | | | | |
| Deaths, N | 0 | 0 | 0 | 0 |
| Patients discontinuing study | 1 | 0 | 0 | 0 |
| drug due to adverse events, | | | | |
| N | | | | |

Noninfectious adverse events, N

| | | | | |
|---|---|---|---|---|
| Injection site reaction | 2 | 0 | 0 | 0 |
| Eosinophilic dermatitis | 1 | 0 | 0 | 0 |
| Fall | 0 | 0 | 1 | 0 |
| Fatigue | 1 | 0 | 0 | 0 |
| Gastrointestinal symptoms | 1 | 1 | 0 | 1 |
| Other orthopedic | 2 | 2 | 1 | 1 |
| injuries/procedures | | | | |
| Bladder malignancy | 0 | 1 | 0 | 0 |
| Prostatic hyperplasia | 0 | 1 | 0 | 0 |
| Facial rash | 0 | 1 | 0 | 0 |
| Conjuctivitis | 3 | 1 | 0 | 0 |

Infectious adverse events, N

| | | | | |
|---|---|---|---|---|
| Upper respiratory tract | 2 | 3 | 2 | 1 |
| infection | | | | |
| Urinary tract infection | 0 | 2 | 0 | 0 |
| Oral herpes | 0 | 0 | 1 | 0 |

The most common AEs were mild upper-respiratory tract infection and mild-to-moderate conjunctivitis. Notably, all conjunctivitis cases were reported in participants with no personal history of AD. Less commonly, injection site reactions, gastrointestinal symptoms, and urinary tract infections were reported, all classified as mild. One patient receiving dupilumab experienced a serious AE (bowel obstruction) that was considered unrelated to study drug by study investigators. AE that led to discontinuation of the study drug occurred in one patient in the drug arm, during the first 24 weeks of the study (reversible drug eruption). In both study arms, there were no clinically relevant changes in hematology and chemistry blood tests or vital signs.

After completing the week 48 part of the study, patients were followed until week 72. Those interested in continuing dupilumab treatment continued to receive dupilumab during this interval. Patients treated with dupilumab until week 72 showed continued improvement in SALT scores, with a least-squares mean change in the SALT score of 29.6 (95% Cl: 21.8-37.5) in the drug-first arm compared to baseline, and of 60.7 (95% Cl: 45.5-76) in the placebo-first arm compared to week 24. Patients discontinuing dupilumab experienced worsening after week 48, with a least-squares mean change in the SALT score of 13 (95% Cl: 0.3-25.7) in the drug-first arm compared to baseline, and of 3.8 (95% Cl: −11.1 to 18.8) in the placebo-first arm compared to week 24 (FIGS. 3A-3D).

### Example 2

Biopsy specimens (˜6 mm) were collected from AA lesions on the scalp of participants enrolled in the study described in Example 1. Samples were collected before treatment, or at 12, 24, and 48 weeks after either dupilumab or placebo administration. Normal skin biopsy samples were collected from the trunk of healthy controls.

RNA was extracted from the samples and sample quality was assessed using FastQC. RNA-Sequencing (RNA-Seq) was performed with the Illumina HiSeq2500 (Illumina, San Diego, California; 100 cycles, single-read sequencing, 8 samples per lane). Samples were aligned to human reference genome using STAR (open source aligner). Mapped sequencing reads were assigned to genomic features using the featureCounts function from Rsubread library. Following standard transformation of counts to log-scale by voom transform, expression values were modeled using mixed-effect models with ethnic group and tissue type as fixed factors and a random effect for each patient. Fold-changes (FCHs) for comparisons of interest were estimated, and hypothesis testing was conducted using contrasts under the general framework for linear models in limma package. P values from the moderated t test were adjusted for multiple hypotheses using the Benjamini-Hochberg procedure, which controls for False Discovery Rate (FDR). Differentially expressed genes (DEGs) were defined by FCH>2.0 or >1.5 where indicated and FDR<0.05. A curated immune gene-subset was elevated using P-values, due to the small sample size. Mean expressions are displayed in a heatmap, where unsupervised clustering was performed using Euclidean distance and average agglomeration criteria.

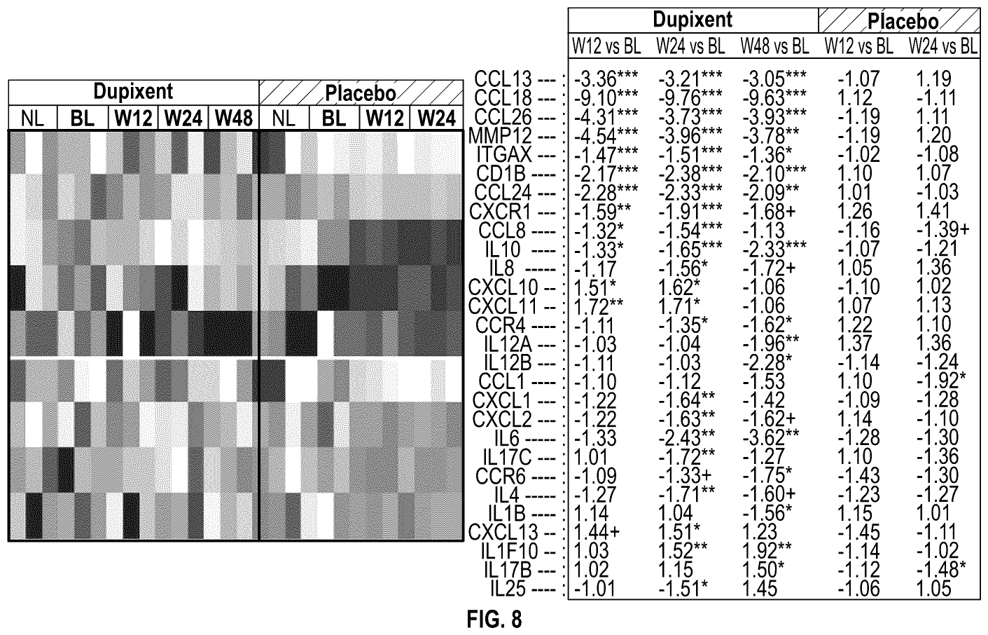

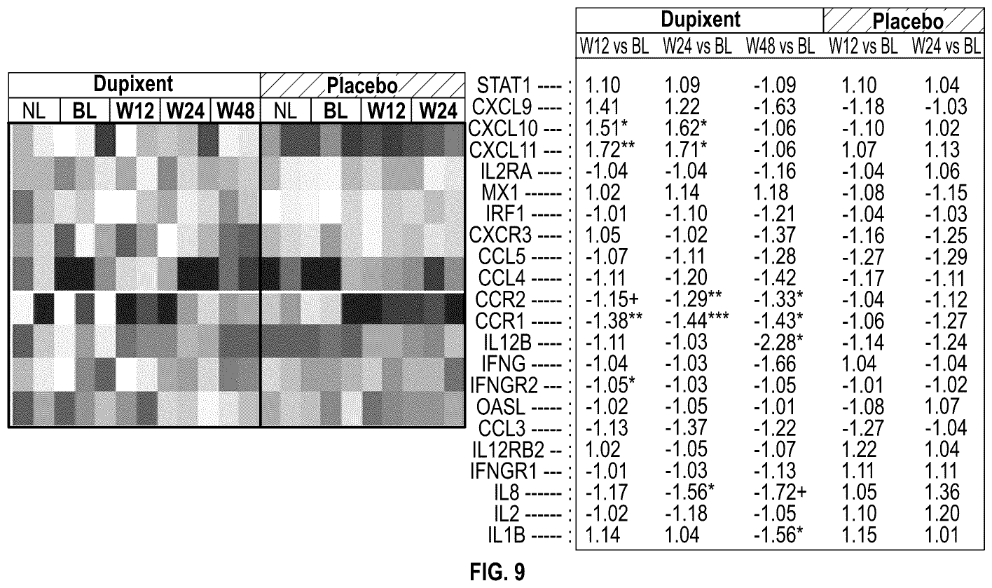

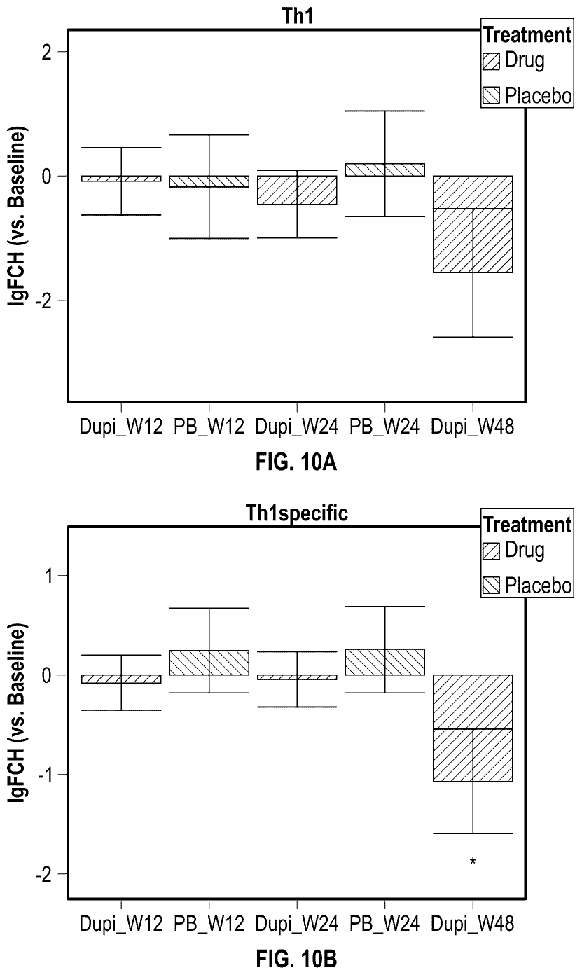

FIG. 8 depicts a heatmap showing differentially expressed immune genes in samples collected from subjects treated with placebo or dupilumab for 0, 12, 24, or 48 weeks. FIG. 9 depicts a heatmap showing differentially expressed immune genes within the Th1 axis in samples collected from subjects treated with placebo or dupilumab for 0, 12, 24, or 48 weeks. qRT-PCR was performed on samples to validate as well as to evaluate key immune molecules that are often below detection limits on RNA-seq. FIGS. 10A-10B show fold-changes of Th1 and Th1 specific immune mediators in samples collected from subjects treated with placebo or dupilumab for 0, 12, 24, or 48 weeks as measured by quantitative real-time PCR.

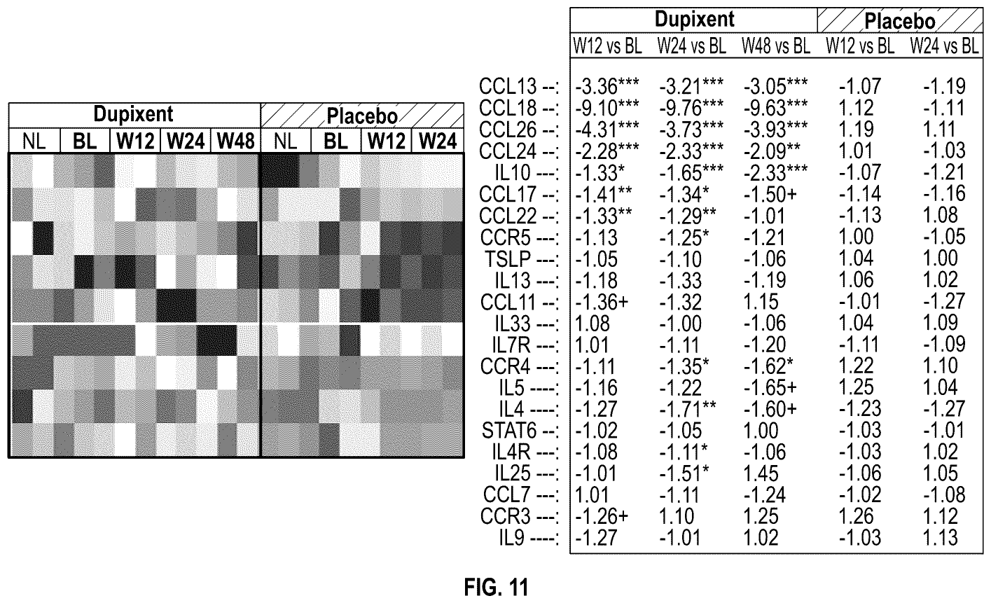

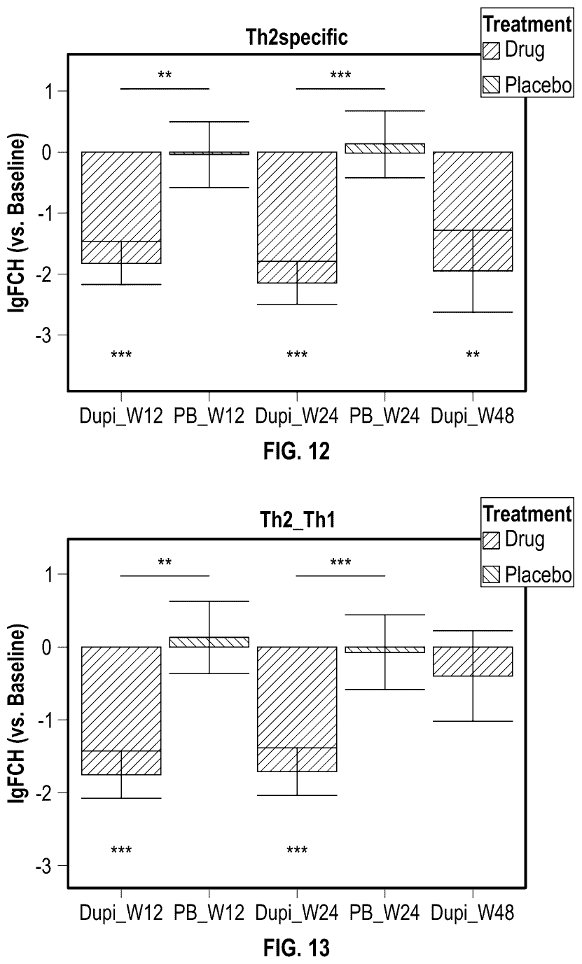

FIG. 11 depicts a heatmap showing differentially expressed immune genes within the Th2 axis in samples collected from subjects treated with placebo or dupilumab for 0, 12, 24, or 48 weeks. FIG. 12 shows fold-changes of Th2/Th1 immune mediators and FIG. 13 shows fold-changes of Th2 specific immune mediators in samples collected from subjects treated with placebo or dupilumab for 0, 12, 24, or 48 weeks as measured by quantitative real-time PCR.

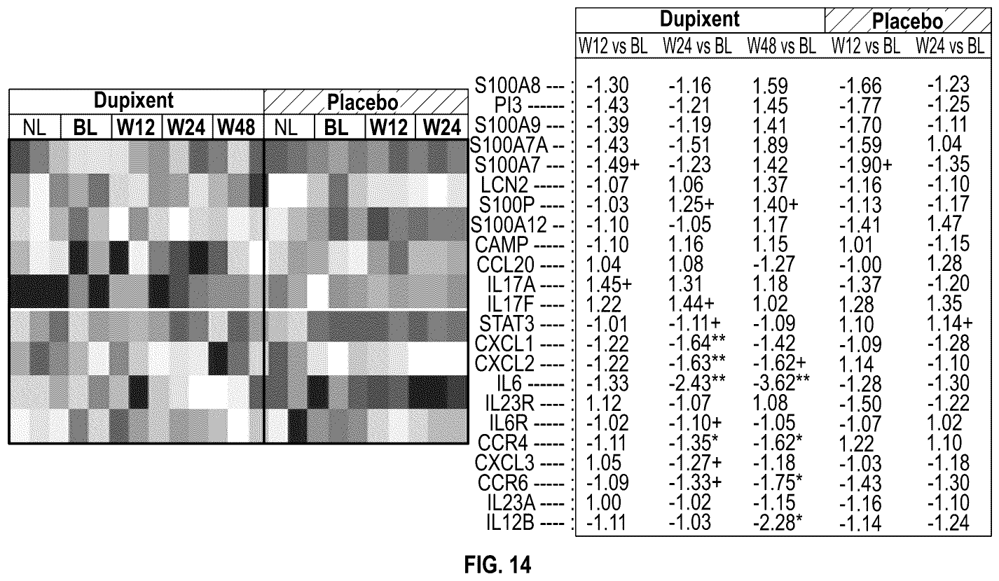

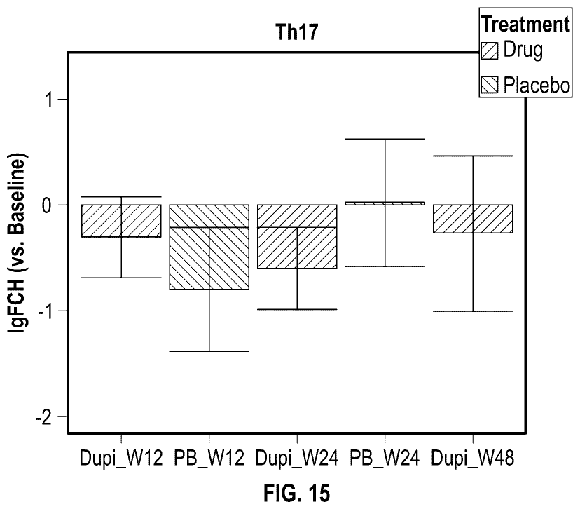

FIG. 14 depicts a heatmap showing differentially expressed immune genes within the Th17 axis in samples collected from subjects treated with placebo or dupilumab for 0, 12, 24, or 48 weeks. FIG. 15 shows fold-changes of Th17 specific immune mediators in samples collected from subjects treated with placebo or dupilumab for 0, 12, 24, or 48 weeks as measured by quantitative real-time PCR.

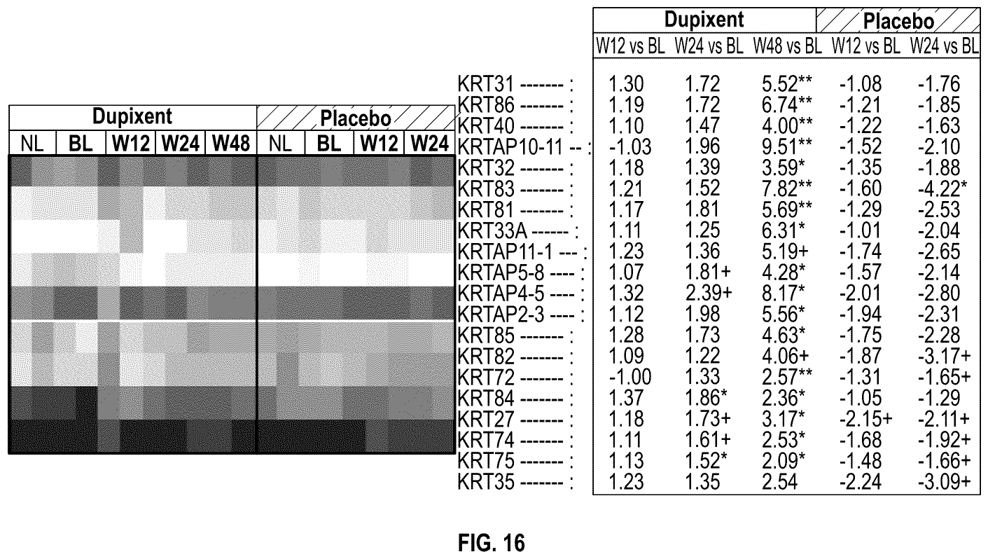

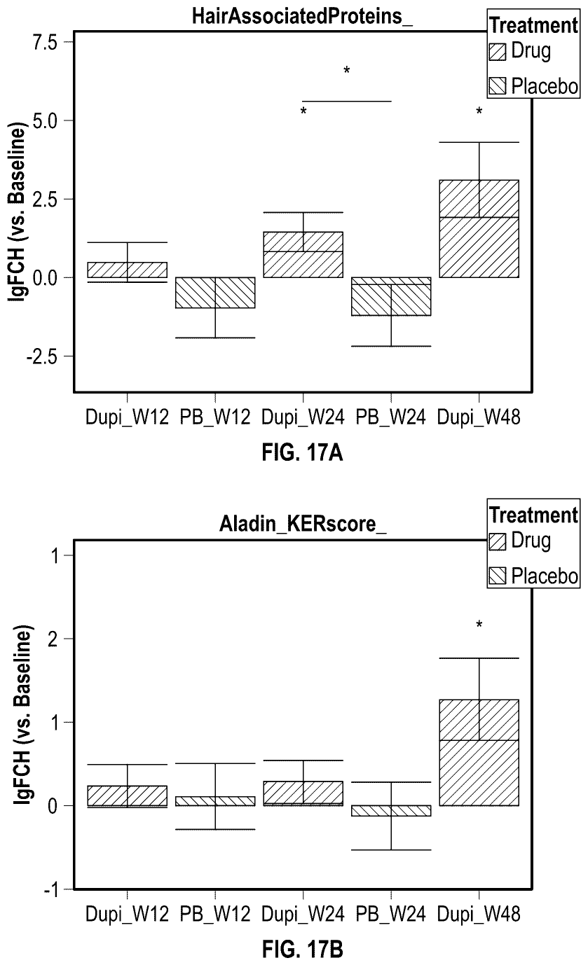

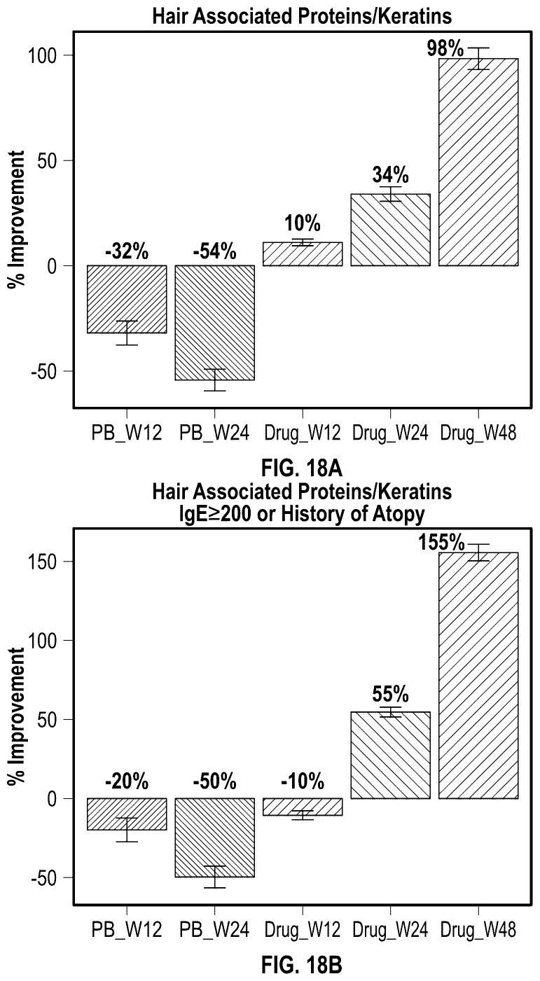

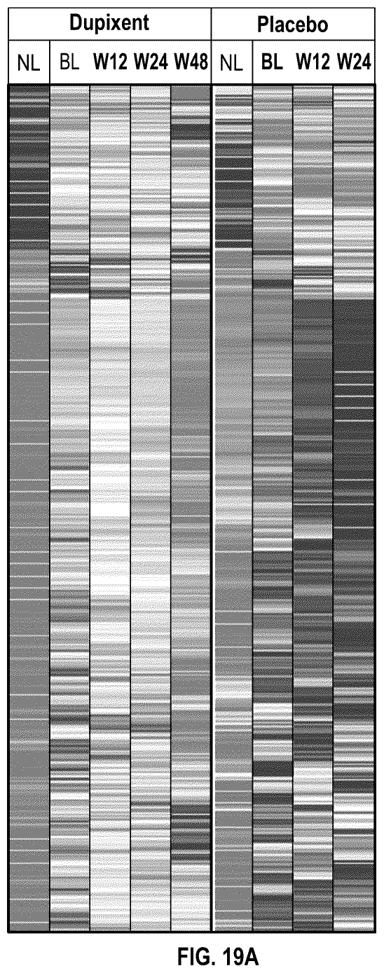

FIG. 16 depicts a heatmap showing differentially expressed genes associated with hair keratins in samples collected from subjects treated with placebo or dupilumab for 0, 12, 24, or 48 weeks. FIGS. 17A-17B show fold-changes of hair protein associated genes in samples collected from subjects treated with placebo or dupilumab for 0, 12, 24, or 48 weeks as measured by quantitative real-time PCR. FIG. 18A shows the number of upregulated hair protein associated genes was significantly increased in subjects treated with dupilumab up to 48 weeks. FIG. 18B shows that in samples from patients with IgE≥200 or a history of atopy, the number of upregulated hair protein associated genes was significantly increased after treatment with dupilumab for 24 and 48 weeks

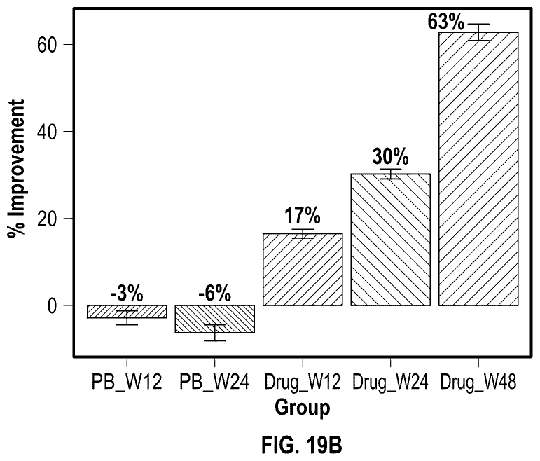

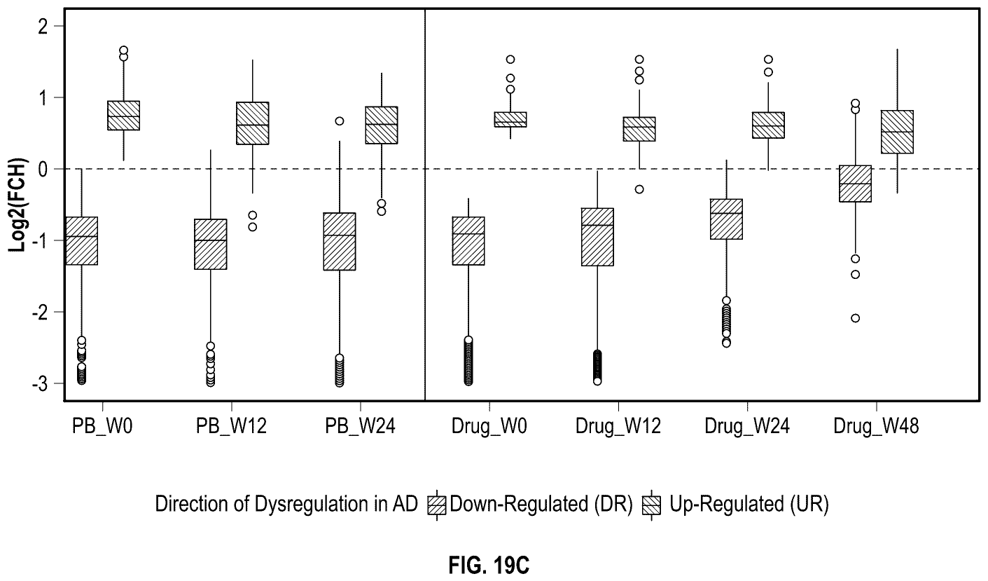

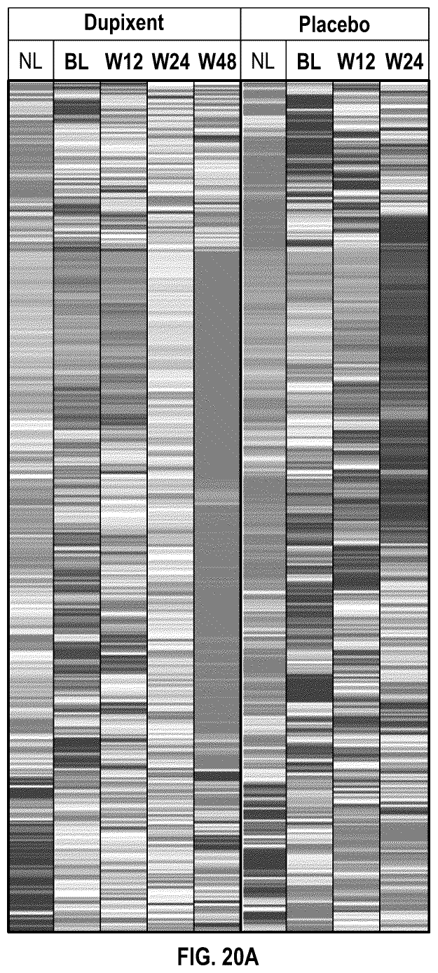

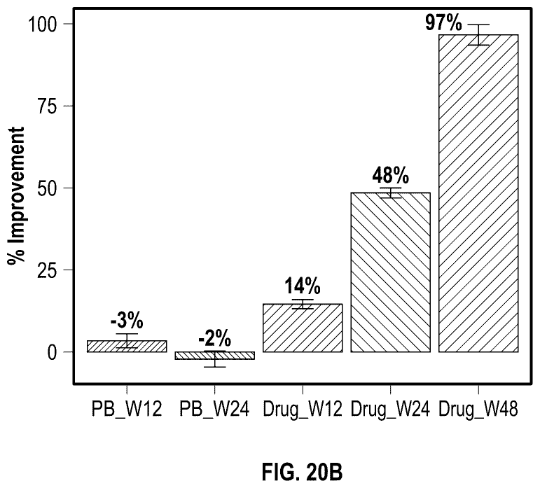

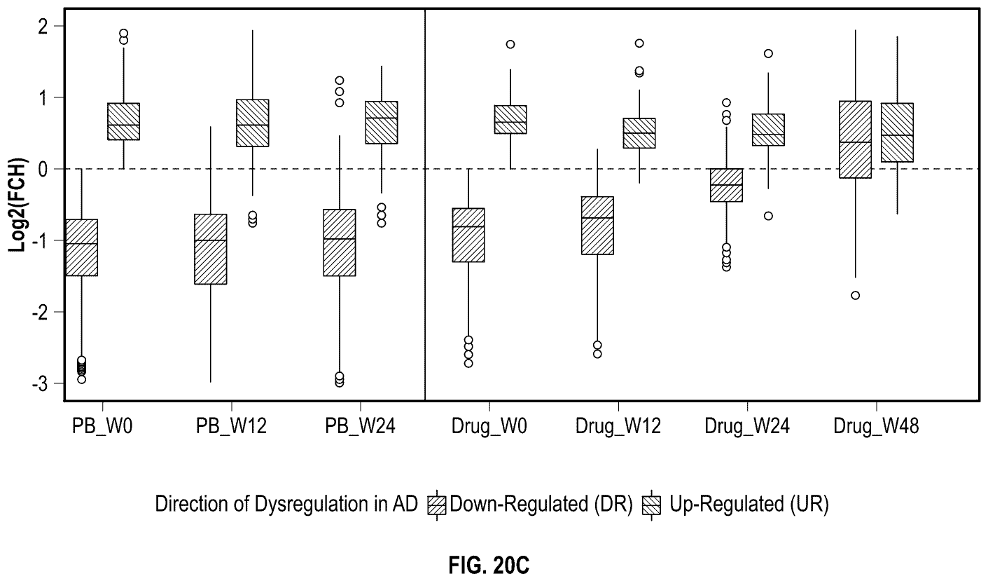

Using criteria of fold-change (FCH)>1.5 and false discovery rate (FDR)<0.05 to define differentially expressed genes (DEGs), 589 DEGs were detected in lesional versus normal skin as depicted in a heatmap in FIG. 19A. FIG. 19B shows improvement in the number of DEGs following dupilumab treatment over time. FIG. 19C shows that treatment with dupilumab for 48 weeks significantly reverses the number of downregulated genes in AA. Next, the same criteria was used to assess DEGs in samples from patients with IgE≥200 or a history of atopy as depicted in a heatmap in FIG. 20A. FIG. 20B shows 97% improvement in the number of DEGs following dupilumab treatment over 48 weeks. FIG. 20C shows that treatment in these patients with dupilumab for 24 or 48 weeks significantly reverses the number of downregulated genes in AA.

### Numbered Embodiments

Notwithstanding the appended claims, the following numbered embodiments are also contemplated herein and form part of the instant disclosure:

1. A method of improving severity of hair loss in a subject, the method comprising: administering to a subject in need thereof an effective amount of an antibody or an antigen-binding fragment thereof, wherein the antibody or antigen-binding fragment thereof specifically binds an interleukin-4 receptor (IL-4R), wherein the subject has or is suspected of having alopecia areata.

2. The method of embodiment 1, wherein the antibody or antigen-binding fragment thereof that specifically binds an interleukin-4 receptor (IL-4R) is dupilumab.

3. The method of embodiment 1 or embodiment 2, wherein the antibody or antigen-binding fragment thereof that specifically binds an interleukin-4 receptor (IL-4R) is formulated in a pharmaceutical composition, which further comprises a pharmaceutically acceptable carrier.

4. The method of any one of embodiments 1-3, wherein the antibody or antigen-binding fragment thereof that specifically binds an interleukin-4 receptor (IL-4R) is administered at a single initial dose comprising 100 mg/kg to 600 mg/kg comprising the antibody or antigen-binding fragment thereof.

5. The method of any one of embodiments 1-4, wherein the subject is a human patient having alopecia areata.

6. The method of embodiment 5, wherein the subject is a human adult patient having alopecia areata.

7. The method of embodiment 6, wherein the antibody or antigen-binding fragment thereof specifically that binds an interleukin-4 receptor (IL-4R) is administered at a single initial dose comprising 600 mg/kg of the antibody or antigen-binding fragment thereof.

8. The method of embodiment 5, wherein the subject is a human child patient having alopecia areata.

9. The method of embodiment 8, wherein the antibody or antigen-binding fragment thereof that specifically binds an interleukin-4 receptor (IL-4R) is administered at a single initial dose comprising 100 mg/kg to 300 mg/kg the antibody or antigen-binding fragment thereof.

10. The method of any one of embodiments 1-9, wherein administration of the single initial dose is followed by one or more secondary doses comprising the antibody or antigen-binding fragment thereof that specifically binds an interleukin-4 receptor (IL-4R).

11. The method of embodiment 10, wherein the secondary doses comprise 100 mg/kg to 600 mg/kg of the antibody or antigen-binding fragment thereof that specifically binds an interleukin-4 receptor (IL-4R).

12. The method of any one of embodiments 1-11, wherein the first secondary dose comprising the antibody or antigen-binding fragment thereof specifically that binds an interleukin-4 receptor (IL-4R) is administered 5 days to 10 days immediately preceding the initial dose.

13. The method of any one of embodiments 1-12, wherein each secondary dose comprising the antibody or antigen-binding fragment thereof specifically that binds an interleukin-4 receptor (IL-4R) is administered by a schedule ranging from once every 5 days to once every 10 days.

14. The method of any one of embodiments 1-13, wherein the antibody or antigen-binding fragment thereof specifically binds an interleukin-4 receptor (IL-4R) is administered to the subject topically, systemically, subcutaneously, intravenously, or intranasally.

15. The method of any one of embodiments 1-14, wherein the subject having or suspected of having alopecia areata has partial to complete hair loss to scalp, face, body or a combination thereof.

16. The method of any one of embodiments 1-15, wherein the subject has undergone or is undergoing another therapy for alopecia areata.

17. The method of embodiment 16, wherein the another therapy for alopecia areata comprises administration of corticosteroids, immunosuppressive agents, potassium channel activators, janus kinase (JAK) inhibitors, interleukin 5 (IL-5) blockers, interleukin 12 (IL-12) blockers, interleukin 12 (IL-13) blockers, interleukin 23 (IL-23) blockers, and Th2 cytokine inhibitors.

18. The method of embodiment 17, wherein the another therapy for alopecia areata comprises administration of at least one Th2 cytokine inhibitor selected from the group comprising OX40, OX40 ligand (OX40L), modulator of Sphingosine-1-phosphate (S1P), modulator of NKG2D, and modulator of thymic stromal lymphopoietin (TSLP).

19. The method of any one of embodiments 1-18, wherein the serum levels of IgE in the subject was assessed prior to administration of the initial dose of the antibody or antigen-binding fragment thereof that specifically binds an interleukin-4 receptor (IL-4R).

20. The method of any one of embodiments 1-19, wherein the serum levels of IgE in the subject was assessed prior to administration of at least one secondary dose of the antibody or antigen-binding fragment thereof that specifically binds an interleukin-4 receptor (IL-4R).

21. A method of treating alopecia areata in a subject that improves severity of hair loss in the subject, the method comprising: (a) selecting a subject having or suspecting of having alopecia areata; (b) measuring serum levels of IgE in the subject, wherein IgE is a biomarker of alopecia areata; (c) administering an initial dose of a pharmaceutical composition comprising dupilumab; wherein the initial dose of the pharmaceutical composition is administered if the serum levels of IgE in the subject are elevated compared to a subject not having or not suspecting of having alopecia areata.

22. The method of embodiment 21, further comprising administering a first secondary dose of a pharmaceutical composition comprising dupilumab 5 days to 10 days immediately preceding the initial dose.

23. The method of embodiment 22, further comprising administering each additional secondary dose of a pharmaceutical composition comprising dupilumab by a schedule ranging from once every 5 days to once every 10 days.

24. The method of embodiment 21, wherein the initial dose of a pharmaceutical composition comprises 100 mg/kg to 600 mg/kg dupilumab.

25. The method of embodiment 22 or embodiment 23, wherein the secondary dose of a pharmaceutical composition comprises 100 mg/kg to 600 mg/kg dupilumab.

26. The method of any one of embodiments 21-25, wherein the subject is a human patient having alopecia areata.

27. The method of embodiment 26, wherein the subject is a human adult patient having alopecia areata.

28. The method of embodiment 27, wherein the initial dose of the pharmaceutical composition comprises 600 mg/kg dupilumab.

29. The method of embodiment 27, wherein the secondary doses of the pharmaceutical composition comprise 300 mg/kg to 600 mg/kg dupilumab.

30. The method of embodiment 26, wherein the subject is a human child patient having alopecia areata.

31. The method of embodiment 30, wherein the initial dose of the pharmaceutical composition comprises 100 mg/kg to 600 mg/kg dupilumab.

32. The method of embodiment 30, wherein the secondary doses of the pharmaceutical composition comprise 100 mg/kg to 600 mg/kg dupilumab.

33. The method of any one of embodiments 21-32, wherein the pharmaceutical composition comprising dupilumab is administered to the subject topically, systemically, subcutaneously, intravenously, or intranasally.

34. The method of any one of embodiments 21-33, wherein the subject having or suspected of having alopecia areata has partial to complete hair loss to scalp, face, body or a combination thereof.

35. The method of any one of embodiments 21-34, further comprising administering at least one other therapy for alopecia areata.

36. The method of embodiment 35, wherein the least one other therapy for alopecia areata comprises administration of corticosteroids, immunosuppressive agents, potassium channel activators, janus kinase (JAK) inhibitors, interleukin 5 (IL-5) blockers, interleukin 12 (IL-12) blockers, interleukin 12 (IL-13) blockers, interleukin 23 (IL-23) blockers, and Th2 cytokine inhibitors.

37. The method of embodiment 36, wherein the another therapy for alopecia areata comprises administration of at least one Th2 cytokine inhibitor selected from the group comprising OX40, OX40 ligand (OX40L), modulator of Sphingosine-1-phosphate (S1P), modulator of NKG2D, and modulator of thymic stromal lymphopoietin (TSLP).

38. The method of any one of embodiments 21-37, further comprising administering an initial dose of at least one JAK inhibitor, wherein the initial dose of the JAK inhibitor is administered if the total IgE serum in the subject are elevated compared to a subject not having or not suspecting of having alopecia areata.

39. The method of any one of embodiments 21-38, wherein the total IgE serum in the subject is measured using at least one electrochemiluminescence immunoassay.

40. A method for determining the likelihood of improving severity of hair loss in a subject in need of at least one treatment for alopecia areata, the method comprising: (a) determining a concentration of a total IgE in a serum sample collected from the subject in need of at least one treatment for alopecia areata; (b) comparing the concentration of total IgE in the serum collected from the subject in need of at least one treatment for alopecia areata to a concentration of total IgE in serum samples collected from a population of normal healthy control subjects; and (c) determining a high likelihood of improving severity of hair loss in the subject in need of at least one treatment for alopecia areata when the total IgE in the serum collected from the subject in need thereof is elevated above a normal healthy concentration of total IgE, wherein the normal healthy concentration of total IgE is the concentration of total IgE in serum samples collected from the population of normal healthy control subjects.

41. The method of embodiment 40 wherein the normal healthy concentration of total IgE is less than 200 IU/ml.

42. The method of either embodiment 40 or embodiment 41, wherein the subject in need of at least one treatment for alopecia areata comprises a human patient having alopecia areata.

43. The method of any one of embodiments 40-42, wherein the subject in need of at least one treatment for alopecia areata comprises administering a steroid, an immunosuppressive agent, a potassium channel activator, a janus kinase (JAK) inhibitor, a Tyrosine Kinase (TYK) inhibitor, an interleukin-4 (IL-4) blocker, an interleukin-4 receptor (IL-4R) blocker, an interleukin 5 (IL-5) blocker, an interleukin 5 receptor (IL-5R) blocker, an interleukin 12/23 p 40 (IL-12/23) blocker, an interleukin 13 (IL-13) blocker, an interleukin 13 receptor (IL-13R) blocker, an interleukin 23 (IL-23) blocker, a type II helper T cell (Th2) cytokine inhibitor, a modulator of Sphingosine-1-phosphate (S1P), a modulator of natural killer group 2 member D (NKG2D), a modulator of thymic stromal lymphopoietin (TSLP), or any combination thereof.

44. The method of any one of embodiments 40-43, wherein the subject in need of at least one treatment for alopecia areata comprises administering cyclosporine A, OX40, OX40 ligand (OX40L), abrocitinib, baricitinib, ritlecitinib, CTP 543, ustekinumab, tralokinumab, lebrikizumab, nemolizumab, upadacitinib, tofacitinib, ruxolitinib, oclacitinib, peficitinib, fedratinib, filgotinib, cerdulatinib, gandotinib, lestaurtinib, momelotinib, pacritinib, BMS-986165, dupilumab, pitrakinra, CBP 201, AK 120, pascolizumab, or any combination thereof.

45. The method of any one of embodiments 40-44, wherein the subject in need of at least one treatment for alopecia areata comprises administering at least dupilumab.

46. The method of any one of embodiments 40-45, wherein the likelihood of improving severity of hair loss comprises at least a 30% improvement from a baseline Severity of Alopecia Tool (SALT) score, wherein the baseline SALT score was measured before the subject was treated with at least one treatment for alopecia areata.

47. The method of any one of embodiments 40-46, wherein the likelihood of improving severity of hair loss comprises up to a 75% to an improvement from a baseline SALT score, wherein the baseline SALT score was measured before the subject was treated with at least one treatment for alopecia areata.

48. The method of any one of embodiments 40-47, wherein the likelihood of improving severity of hair loss comprises at least a 30% improvement from a baseline SALT score after 8 to 72 weeks of alopecia areata treatment, wherein the baseline SALT score was measured before the subject was treated with at least one treatment for alopecia areata.

49. The method of any one of embodiments 40-48, wherein the likelihood of improving severity of hair loss comprises at least a 50% improvement from a baseline SALT score after 12 to 72 weeks of alopecia areata treatment, wherein the baseline SALT score was measured before the subject was treated with at least one treatment for alopecia areata.

50. The method of any one of embodiments 40-49, wherein the likelihood of improving severity of hair loss comprises at least a 75% improvement from a baseline SALT score after 20 to 72 weeks of alopecia areata treatment, wherein the baseline SALT score was measured before the subject was treated with at least one treatment for alopecia areata.

51. A method for determining the likelihood of improving severity of hair loss in a subject to be treated with dupilumab, the method comprising: (a) determining a concentration of a total IgE in a serum sample collected from the subject to be treated with dupilumab; (b) comparing the concentration of total IgE in the serum collected from the subject to be treated with dupilumab to a concentration of total IgE in serum samples collected from a population of normal healthy control subjects; and (c) determining a high likelihood of improving severity of hair loss when the total IgE in the serum collected from the subject to be treated with dupilumab is elevated above a normal healthy concentration of total IgE, wherein the normal healthy concentration of total IgE is the concentration of total IgE in serum samples collected from the population of normal healthy control subjects.

52. The method of embodiment 51 wherein the normal healthy concentration of total IgE is less than 200 IU/ml.

53. The method of either embodiment 51 or embodiment 52, wherein the likelihood of improving severity of hair loss comprises at least a 30% improvement from a baseline Severity of Alopecia Tool (SALT) score, wherein the baseline SALT score was measured before the subject was treated with dupilumab.

54. The method of any one of embodiments 51-53, wherein the likelihood of improving severity of hair loss comprises up to a 75% to a improvement from a baseline SALT score, wherein the baseline SALT score was measured before the subject was treated with dupilumab.

55. The method of any one of embodiments 51-54, wherein the likelihood of improving severity of hair loss comprises at least a 30% improvement from a baseline SALT score after 8 to 72 weeks of dupilumab treatment, wherein the baseline SALT score was measured before the subject was treated with dupilumab.

56. The method of any one of embodiments 51-55, wherein the likelihood of improving severity of hair loss comprises at least a 50% improvement from a baseline SALT score after 12 to 72 weeks of dupilumab treatment, wherein the baseline SALT score was measured before the subject was treated with dupilumab.

57. The method of any one of embodiments 51-55, wherein the likelihood of improving severity of hair loss comprises at least a 75% improvement from a baseline SALT score after 20 to 72 weeks of dupilumab treatment, wherein the baseline SALT score was measured before the subject was treated with dupilumab.

58. The method of any one of embodiments 51-57, wherein the subject to be treated with dupilumab comprises a human patient having alopecia areata.

59. The method of embodiment 58, wherein the subject to be treated with dupilumab is a human adult patient having alopecia areata or a human child patient having alopecia areata.

60. A method of improving severity of hair loss in a subject, the method comprising: measuring a total IgE serum in the subject; administering to a subject in need thereof an effective amount of an antibody or an antigen-binding fragment thereof, wherein the antibody or antigen-binding fragment thereof specifically binds an interleukin-4 receptor (IL-4R), wherein the subject has or is suspected of having alopecia areata and the total IgE serum equal to or greater than 200 IU/ml.

61. The method of embodiment 60, wherein the antibody or antigen-binding fragment thereof that specifically binds an interleukin-4 receptor (IL-4R) is dupilumab.

62. The method of embodiment 60 or embodiment 61, wherein the antibody or antigen-binding fragment thereof that specifically binds an interleukin-4 receptor (IL-4R) is formulated in a pharmaceutical composition, which further comprises a pharmaceutically acceptable carrier.

63. The method of any one of embodiments 60-62, wherein the antibody or antigen-binding fragment thereof that specifically binds an interleukin-4 receptor (IL-4R) is administered at a single initial dose comprising 100 mg/kg to 600 mg/kg comprising the antibody or antigen-binding fragment thereof.

64. The method of any one of embodiments 60-63, wherein the subject is a human patient having alopecia areata.

65. The method of embodiment 64, wherein the subject is a human adult patient having alopecia areata.

66. The method of embodiment 65, wherein the antibody or antigen-binding fragment thereof specifically that binds an interleukin-4 receptor (IL-4R) is administered at a single initial dose comprising 600 mg/kg of the antibody or antigen-binding fragment thereof.

67. The method of embodiment 64, wherein the subject is a human child patient having alopecia areata.

68. The method of embodiment 67, wherein the antibody or antigen-binding fragment thereof that specifically binds an interleukin-4 receptor (IL-4R) is administered at a single initial dose comprising 100 mg/kg to 300 mg/kg the antibody or antigen-binding fragment thereof.

69. The method of any one of embodiments 60-68, wherein administration of the single initial dose is followed by one or more secondary doses comprising the antibody or antigen-binding fragment thereof that specifically binds an interleukin-4 receptor (IL-4R).

70. The method of embodiment 69, wherein the secondary doses comprise 100 mg/kg to 600 mg/kg of the antibody or antigen-binding fragment thereof that specifically binds an interleukin-4 receptor (IL-4R).

71. The method of any one of embodiments 60-70, wherein the first secondary dose comprising the antibody or antigen-binding fragment thereof specifically that binds an interleukin-4 receptor (IL-4R) is administered 5 days to 10 days immediately preceding the initial dose.

72. The method of any one of embodiments 60-71, wherein each secondary dose comprising the antibody or antigen-binding fragment thereof specifically that binds an interleukin-4 receptor (IL-4R) is administered by a schedule ranging from once every 5 days to once every 10 days.

73. The method of any one of embodiments 60-72, wherein the antibody or antigen-binding fragment thereof specifically binds an interleukin-4 receptor (IL-4R) is administered to the subject topically, systemically, subcutaneously, intravenously, or intranasally.

74. The method of any one of embodiments 60-73, wherein the subject having or suspected of having alopecia areata has partial to complete hair loss to scalp, face, body or a combination thereof.

75. The method of any one of embodiments 60-74, wherein the subject has undergone or is undergoing another therapy for alopecia areata.

76. The method of embodiment 75, wherein the another therapy for alopecia areata comprises administration of corticosteroids, immunosuppressive agents, potassium channel activators, janus kinase (JAK) inhibitors, interleukin 5 (IL-5) blockers, interleukin 12 (IL-12) blockers, interleukin 12 (IL-13) blockers, interleukin 23 (IL-23) blockers, and Th2 cytokine inhibitors.

77. The method of embodiment 76, wherein the another therapy for alopecia areata comprises administration of at least one Th2 cytokine inhibitor selected from the group comprising OX40, OX40 ligand (OX40L), modulator of Sphingosine-1-phosphate (S1P), modulator of NKG2D, and modulator of thymic stromal lymphopoietin (TSLP).

78. The method of any one of embodiments 60-77, wherein the total IgE serum in the subject was measured prior to administration of the initial dose of the antibody or antigen-binding fragment thereof that specifically binds an interleukin-4 receptor (IL-4R).

79. The method of any one of embodiments 60-78, wherein the total IgE serum in the subject was measured prior to administration of at least one secondary dose of the antibody or antigen-binding fragment thereof that specifically binds an interleukin-4 receptor (IL-4R).

80. A method of treating alopecia areata in a subject that improves severity of hair loss in the subject, the method comprising: (a) selecting a subject having or suspecting of having alopecia areata; (b) measuring total IgE serum in the subject, wherein IgE is a biomarker of alopecia areata; (c) administering an initial dose of a pharmaceutical composition comprising dupilumab; wherein the initial dose of the pharmaceutical composition is administered if the total IgE serum in the subject are elevated compared to a subject not having or not suspecting of having alopecia areata.

81. The method of embodiment 80, further comprising administering a first secondary dose of a pharmaceutical composition comprising dupilumab 5 days to 10 days immediately preceding the initial dose.

82. The method of embodiment 81, further comprising administering each additional secondary dose of a pharmaceutical composition comprising dupilumab by a schedule ranging from once every 5 days to once every 10 days.

83. The method of embodiment 82, wherein the initial dose of a pharmaceutical composition comprises 100 mg/kg to 600 mg/kg dupilumab.

84. The method of embodiment 81 or embodiment 82, wherein the secondary dose of a pharmaceutical composition comprises 100 mg/kg to 600 mg/kg dupilumab.

85. The method of any one of embodiments 80-84, wherein the subject is a human patient having alopecia areata.

86. The method of embodiment 85, wherein the subject is a human adult patient having alopecia areata.

87. The method of embodiment 86, wherein the initial dose of the pharmaceutical composition comprises 600 mg/kg dupilumab.

88. The method of embodiment 86, wherein the secondary doses of the pharmaceutical composition comprise 300 mg/kg to 600 mg/kg dupilumab.

89. The method of embodiment 85, wherein the subject is a human child patient having alopecia areata.

90. The method of embodiment 89, wherein the initial dose of the pharmaceutical composition comprises 100 mg/kg to 600 mg/kg dupilumab.

91. The method of embodiment 89, wherein the secondary doses of the pharmaceutical composition comprise 100 mg/kg to 600 mg/kg dupilumab.

92. The method of any one of embodiments 80-91, wherein the pharmaceutical composition comprising dupilumab is administered to the subject topically, systemically, subcutaneously, intravenously, or intranasally.

93. The method of any one of embodiments 80-92, wherein the subject having or suspected of having alopecia areata has partial to complete hair loss to scalp, face, body or a combination thereof.

94. The method of any one of embodiments 80-93, further comprising administering at least one other therapy for alopecia areata.

95. The method of embodiment 94, wherein the least one other therapy for alopecia areata comprises administration of corticosteroids, immunosuppressive agents, potassium channel activators, janus kinase (JAK) inhibitors, interleukin 5 (IL-5) blockers, interleukin 12 (IL-12) blockers, interleukin 12 (IL-13) blockers, interleukin 23 (IL-23) blockers, and Th2 cytokine inhibitors.

96. The method of embodiment 95, wherein the another therapy for alopecia areata comprises administration of at least one Th2 cytokine inhibitor selected from the group comprising OX40, OX40 ligand (OX40L), modulator of Sphingosine-1-phosphate (S1P), modulator of NKG2D, and modulator of thymic stromal lymphopoietin (TSLP).

97. The method of any one of embodiments 80-96, wherein the total IgE serum in the subject is measured using at least one electrochemiluminescence immunoassay.

1.-20. (canceled)

21. A method of improving severity of hair loss in a subject, the method comprising: administering to a subject in need thereof an effective amount of dupilumab, wherein the subject has or is suspected of having alopecia areata.

22. The method of claim 21, wherein dupilumab is formulated in a pharmaceutical composition, which further comprises a pharmaceutically acceptable carrier.

23. The method of claim 21, wherein dupilumab is administered at a single initial dose comprising 100 mg/kg to 600 mg/kg dupilumab.

24. The method of claim 21, wherein the subject is a human patient having alopecia areata.

25. The method of claim 24, wherein the subject is a human adult patient having alopecia areata and dupilumab is administered a single initial dose comprising 600 mg/kg dupilumab; or the subject is a human child patient having alopecia areata and dupilumab is administered at a single initial dose comprising 100 mg/kg to 300 mg/kg dupilumab.

26. The method of claim 25, wherein administration of the single initial dose is followed by one or more secondary doses comprising dupilumab.

27. The method of claim 26, wherein the secondary doses comprise 100 mg/kg to 600 mg/kg of dupilumab.

28. The method of claim 26, wherein the one or more secondary doses comprising dupilumab are administered 5 days to 10 days after the initial dose.

29. The method of claim 26, wherein the one or more secondary doses comprising dupilumab are administered once every 5 days to once every 10 days after administration of the single initial dose.

30. The method of claim 25, wherein the serum levels of IgE in the subject was assessed prior to administration of the initial dose of dupilumab.

31. A method of treating alopecia areata in a subject that improves severity of hair loss in the subject, the method comprising:
(a) selecting a subject having or suspecting of having alopecia areata;
(b) measuring total IgE serum in the subject, wherein IgE is a biomarker of alopecia areata;
(c) administering an initial dose of a pharmaceutical composition comprising dupilumab; wherein the initial dose of the pharmaceutical composition is administered if the total IgE serum in the subject is elevated compared to a subject not having or not suspecting of having alopecia areata.

32. The method of claim 31, further comprising administering a first secondary dose of a pharmaceutical composition comprising dupilumab 5 days to 10 days after the initial dose.

33. The method of claim 31, wherein the initial dose of a pharmaceutical composition comprises 100 mg/kg to 600 mg/kg dupilumab.

34. A method for determining the likelihood of improving severity of hair loss in a subject in need of at least one treatment for alopecia areata, the method comprising:
(a) determining a concentration of a total IgE in a serum sample collected from the subject in need of at least one treatment for alopecia areata;
(b) comparing the concentration of total IgE in the serum collected from the subject in need of at least one treatment for alopecia areata to a concentration of total IgE in serum samples collected from a population of normal healthy control subjects; and
(c) determining a high likelihood of improving severity of hair loss in the subject in need of at least one treatment for alopecia areata when the total IgE in the serum collected from the subject in need thereof is elevated above a normal healthy concentration of total IgE, wherein the normal healthy concentration of total IgE is the concentration of total IgE in serum samples collected from the population of normal healthy control subjects.

35. The method of claim 34 wherein the normal healthy concentration of total IgE is less than 200 IU/ml.

36. The method of either claim 34, wherein the subject in need of at least one treatment for alopecia areata comprises administering a steroid, an immunosuppressive agent, a potassium channel activator, a j anus kinase (JAK) inhibitor, a Tyrosine Kinase (TYK) inhibitor, an interleukin-4 (IL-4) blocker, an interleukin-4 receptor (IL-4R) blocker, an interleukin 5 (IL-5) blocker, an interleukin 5 receptor (IL-5R) blocker, an interleukin 12/23 p 40 (IL-12/23) blocker, an interleukin 13 (IL-13) blocker, an interleukin 13 receptor (IL-13R) blocker, an interleukin 23 (IL-23) blocker, a type II helper T cell (Th2) cytokine inhibitor, a modulator of Sphingosine-1-phosphate (SIP), a modulator of natural killer group 2 member D (NKG2D), a modulator of thymic stromal lymphopoietin (TSLP), or any combination thereof.
